(19) United States
(12) Patent Application Publication
(10) Pub. No.: US 2026/0176969 A1
(43) Pub. Date: Jun. 25, 2026

(54) MANAGING METHANE LEAKS WITH MINIMAL IMPACT ON PRODUCTIVITY

(71) Applicant: International Business Machines Corporation, Armonk, NY (US)

(72) Inventors: Sarbajit Kumar Rakshit, Kolkata (IN); Jagabondhu Hazra, Bangalore (IN); Manikandan Padmanaban, Chennai (IN)

(21) Appl. No.: 18/990,843

(22) Filed: Dec. 20, 2024

(51) Int. Cl. E21F 7/00; G01N 33/00; G06Q 50/02

(52) U.S. Cl. CPC E21F 7/00; G06Q 50/02; G01N 33/0047

(57) ABSTRACT

A computer-implemented method, system, and computer program product for managing methane leaks in mining operations in a manner that prevents methane explosions with minimal impact on productivity. Levels of concentration of methane are monitored in the air using methane sensors, such as at a mining facility. Upon identifying a level of concentration of methane in the air from a methane sensor that exceeds a threshold value, which may be user-designated, a virtual fence is created around the methane sensor to identify an area where the level of concentration of methane in the air exceeds the threshold value. A methane removal strategy is then developed for removing the methane within the virtual fence, such as based on the cost of deploying drones with oxygen, temperature, and catalyst units. After developing a methane removal strategy for removing the methane within the virtual fence, the methane remove strategy is implemented.

### TECHNICAL FIELD

The present disclosure relates generally to mining operations, and more particularly to managing methane leaks involved in mining operations (e.g., coal mining operations) with minimal impact on productivity.

### BACKGROUND

Mining operations are the actions involved in extracting minerals from the earth, including the development, transportation, and processing of minerals. For example, the development of minerals involves geological surveys, prospecting, drilling, and designing. Transportation of minerals involves moving minerals to another location. The processing of minerals involves processes, such as concentrating, milling, evaporation, etc. Mining operations may also involve extraction (e.g., blasting, loading, and hauling) and disposal (e.g., disposing of refuse from underground mining). Mining operations can take place on the surface or underground. They can include open mining, in situ mining, in situ leach mining, and surface operations.

### SUMMARY

In one embodiment of the present disclosure, a computer-implemented method for managing methane leaks comprises monitoring levels of concentration of methane in air using methane sensors. The method further comprises monitoring identifying a level of concentration of methane in the air from a first methane sensor that exceeds a first threshold value. The method additionally comprises creating a virtual fence around the first methane sensor to identify an area where the level of concentration of methane in the air exceeds the first threshold value. Furthermore, the method comprises developing a methane removal strategy for removing methane within the virtual fence. Additionally, the method comprises implementing the methane removal strategy for removing methane within the virtual fence.

Other forms of the embodiment of the computer-implemented method described above are in a system and in a computer program product.

The foregoing has outlined rather generally the features and technical advantages of one or more embodiments of the present disclosure in order that the detailed description of the present disclosure that follows may be better understood. Additional features and advantages of the present disclosure will be described hereinafter which may form the subject of the claims of the present disclosure.

### BRIEF DESCRIPTION OF THE DRAWINGS

A better understanding of the present disclosure can be obtained when the following detailed description is considered in conjunction with the following drawings, in which:

FIG. 1 illustrates an embodiment of the present disclosure of a communication system for practicing the principles of the present disclosure;

FIG. 2 illustrates the internal components of a mining machine, such as an autonomous mining machine, in accordance with an embodiment of the present disclosure;

FIG. 3 illustrates a perspective view of the drone in accordance with an embodiment of the present disclosure;

FIG. 4 illustrates the hardware configuration of the drone in accordance with an embodiment of the present disclosure;

FIG. 5 is a diagram of the software components of the methane leak manager used to manage methane leaks at a mining facility in a manner that prevents methane explosions with minimal impact on productivity in accordance with an embodiment of the present disclosure;

FIG. 6 illustrates monitoring for methane leaks, such as at a mining facility, in accordance with an embodiment of the present disclosure;

FIG. 7 illustrates an embodiment of the present disclosure of the hardware configuration of the methane leak manager which is representative of a hardware environment for practicing the present disclosure;

FIG. 8 is a flowchart of a method for training a machine learning model for altering the work order schedules of those mining machines located within or surrounding a virtual fence with a risk of setting a fire that exceeds a threshold value in accordance with an embodiment of the present disclosure; and

FIG. 9 is a flowchart of a method for managing methane leaks in mining operations in a manner that prevents methane explosions with minimal impact on productivity in accordance with an embodiment of the present disclosure.

### DETAILED DESCRIPTION

As stated above, mining operations are the actions involved in extracting minerals from the earth, including the development, transportation, and processing of minerals. For example, the development of minerals involves geological surveys, prospecting, drilling, and designing. Transportation of minerals involves moving minerals to another location. The processing of minerals involves processes, such as concentrating, milling, evaporation, etc. Mining operations may also involve extraction (e.g., blasting, loading, and hauling) and disposal (e.g., disposing of refuse from underground mining). Mining operations can take place on the surface or underground. They can include open mining, in situ mining, in situ leach mining, and surface operations.

Such mining operations may involve the leakage of methane (CH₄), such as from coal seams and surrounding rock strata. For example, methane may be released from coal seams. When coal seams are fractured during mining, the methane trapped under pressure is released. In surface mines, drainage systems are a source of methane emissions. Furthermore, methane can seep out during the processing, storage, and transport of coal in underground mines. Additionally, methane can be released from abandoned mines.

Methane is a greenhouse gas that is explosive when mixed with air so it poses a safety risk. An explosion may be set off by an electrostatic spark creating a huge increase in gas pressure. Such an electrostatic spark may be caused by the mining machines operating in the area where the concentration level of methane has reached an unsafe level.

Such methane explosions can lead to mine evacuations, shutdowns, and stoppages, which can cause significant delays in production and loss of revenue.

Unfortunately, there is not currently a means for managing methane leaks in mining operations in a manner that prevents methane explosions with minimal impact on productivity.

The embodiments of the present disclosure provide a means for managing methane leaks in mining operations in a manner that prevents methane explosions with minimal impact on productivity. In one embodiment, levels of concentration of methane are monitored in the air using methane sensors, such as at a mining facility. A mining facility, as used herein, is a place that extracts, mills, and processes minerals into a marketable form. It may include buildings, equipment, machinery, and other infrastructure. Upon identifying a level of concentration of methane in the air from a methane sensor that exceeds a threshold value, which may be user-designated, a virtual fence is created around the methane sensor to identify an area where the level of concentration of methane in the air exceeds the threshold value. A virtual fence, as used herein, refers to an invisible border, which marks the area where the level of concentration of methane in the air exceeds the threshold value. A methane removal strategy (e.g., deploying drones to remove methane within the virtual fence using diffusion or adsorption, using a ventilation-based system) is then developed for removing the methane within the virtual fence, such as based on the cost of deploying drones with oxygen, temperature, and catalyst units. Oxygen is used by drones to remove methane because it combines with methane to form carbon dioxide, a less potent greenhouse gas. Temperature is important for removing methane because it affects how the gas breaks down and the energy required for the process. For example, methane decomposition is an endothermic process (i.e., requires high temperature to break down the gas). Furthermore, the energy demand for a methane removal process is affected by the temperature at which the reaction occurs. Catalysts are needed to remove methane because they can accelerate the oxidation of methane by air, turning it into less harmful substances, such as carbon dioxide. Examples of catalysts include, but are not limited to, aluminosilicate minerals, palladium-based catalysts, mechanochemically prepared catalysts, etc. After developing a methane removal strategy for removing the methane within the virtual fence, the methane remove strategy is implemented.

Furthermore, in connection with developing a methane removal strategy, one or more mining machines are identified within and surrounding the virtual fence with a risk of setting a fire that exceeds a threshold value. A mining machine, as used herein, refers to tools used to extract raw materials from the earth, and include a variety of types of equipment, such as an excavator, a crusher, a wheel loader, a blasthole drill, a bucket-wheel excavator, a dozer, a dragline excavator, a grader, a highwall miner, a mining truck, a continuous miner, etc. In one embodiment, such mining machines with a risk of setting a fire are identified using digital twins of such mining machines. A digital twin, as used herein, refers to a virtual model of the mining machine that may use real-time data to simulate how it behaves in the real world.

In one embodiment, the work order schedule of the identified mining machines (those mining machines identified with a risk of setting a fire that exceeds a threshold value) are altered by having the previously scheduled operations of the identified mining machine(s) that pose a risk of setting a fire being performed by an alternative mining machine(s). In one embodiment, the work order schedule of the identified mining machines are altered based on the business criticality of the identified mining machines and the available alternative mining machines, the functionality of the identified mining machines and the available alternative mining machines, the current work order schedule of the available alternative mining machines, the risk of setting a fire by the available alternative mining machines, etc.

In one embodiment, upon the level of concentration of methane being reduced to a safe level, the work order schedule for the identified mining machines that previously posed a risk of setting a fire is altered to perform the operation(s) originally assigned to the identified mining machines that may not have been completed by the alternative mining machines.

In this manner, methane leaks in mining operations are managed in a manner that prevents methane explosions with minimal impact on productivity. A further discussion regarding these and other features is provided below.

In the following description, numerous specific details are set forth to provide a thorough understanding of the present disclosure. However, it will be apparent to those skilled in the art that the present disclosure may be practiced without such specific details. In other instances, well-known circuits have been shown in block diagram form in order not to obscure the present disclosure in unnecessary detail. For the most part, details considering timing considerations and the like have been omitted inasmuch as such details are not necessary to obtain a complete understanding of the present disclosure and are within the skills of persons of ordinary skill in the relevant art.

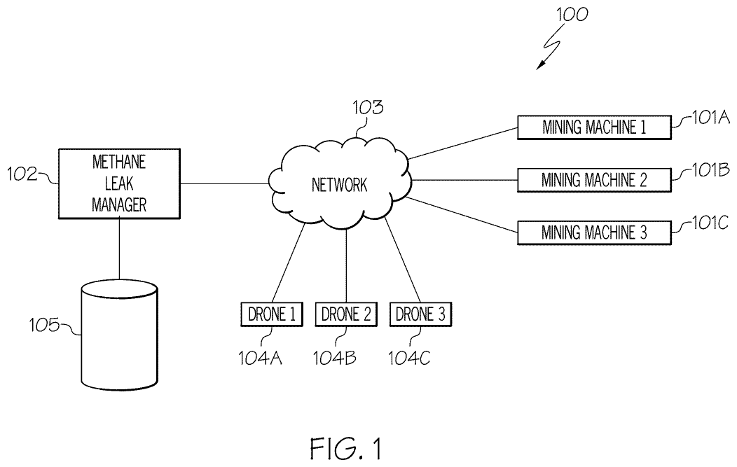

Referring now to the Figures in detail, FIG. 1 illustrates an embodiment of the present disclosure of a communication system 100 (or simply “system”) for practicing the principles of the present disclosure. System 100 includes mining machines 101A-101C (identified as “mining machine 1,” “mining machine 2,” and “mining machine 3,” respectively, in FIG. 1) connected to a methane leak manager 102 via a network 103.

Mining machines 101A-101C may collectively or individually be referred to as mining machines 101 or mining machine 101, respectively. While FIG. 1 illustrates system 100 including three mining machines 101, system 100 may include any number of mining machines 101.

A mining machine 101, as used herein, refers to tools used to extract raw materials from the earth, and include a variety of types of equipment, such as an excavator, a crusher, a wheel loader, a blasthole drill, a bucket-wheel excavator, a dozer, a dragline excavator, a grader, a highwall miner, a mining truck, a continuous miner, etc.

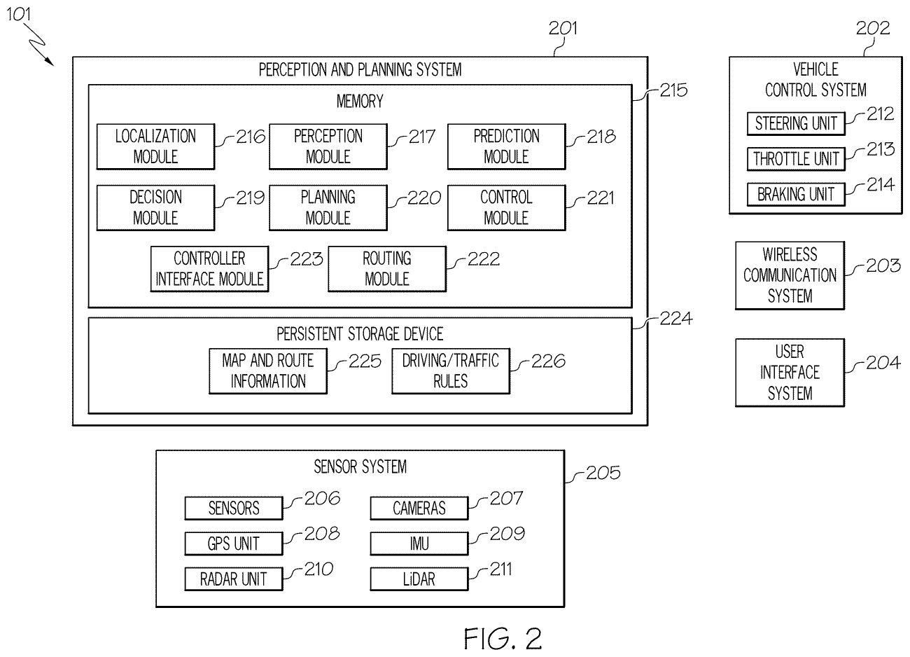

In one embodiment, mining machine 101 is autonomous. An autonomous mining machine, as used herein, refers to a mining machine capable of sensing its environment and operating without human involvement. A description of the internal components of such an embodiment of mining machine 101 is provided below in connection with FIG. 2. In one embodiment, mining machine 101 is controlled by a human operator.

System 100 further includes drones 104A-104C (identified as “drone 1,” “drone 2,” and “drone 3,” respectively, in FIG. 1) connected to methane leak manager 102 via network 103.

Drones 104A-104C may collectively or individually be referred to as drones 104 or drone 104, respectively. While FIG. 1 illustrates system 100 including three drones 104, system 100 may include any number of drones 104.

A “drone 104,” as used herein, refers an unmanned aerial vehicle used to remove methane, such as at a mining facility. A mining facility, as used herein, is a place that extracts, mills, and processes minerals into a marketable form. It may include buildings, equipment, machinery, and other infrastructure.

In one embodiment, drones 104 are configured to remove methane within a virtual fence. A virtual fence, as used herein, refers to an invisible border, which marks the area where the level of concentration of methane in the air exceeds a threshold value, such as an unsafe level. In one embodiment, drones 104 are equipped with oxygen, temperature, and catalyst units. Oxygen units are used by drones 104 to remove methane because it combines with methane to form carbon dioxide, a less potent greenhouse gas. Temperature units are used by drones 104 for removing methane because it affects how the gas breaks down and the energy required for the process. For example, methane decomposition is an endothermic process (i.e., requires high temperature to break down the gas) Without a catalyst, the decomposition process requires a temperature of around 1,200° C. Furthermore, the energy demand for a methane removal process is affected by the temperature at which the reaction occurs. Catalyst units are used by drones 104 to remove methane because they can accelerate the oxidation of methane by air, turning it into less harmful substances, such as carbon dioxide. Examples of catalysts include, but are not limited to, aluminosilicate minerals, palladium-based catalysts, mechanochemically prepared catalysts, etc.

In one embodiment, drones 104 are used to remove methane within a virtual fence via diffusion using metal catalysts, such as zeolite, copper-zinc oxide, etc., or photocatalysts, such as tin oxide, etc. Removing methane by diffusion, as used herein, refers to the process where methane gas moves from a region of high concentration (e.g., area within the virtual fence) to a region of low concentration (e.g., atmosphere) through the physical process of diffusion thereby allowing the methane to disperse and gradually dissipate into the surrounding air.

In one embodiment, drones 104 are used to remove methane within a virtual fence via adsorption. Removing methane by adsorption, as used herein, refers to utilizing adsorbents to remove methane. Examples of such adsorbents include, but are not limited to, metal-organic frameworks, biochar, nano-size zeolites, zeolites, etc.

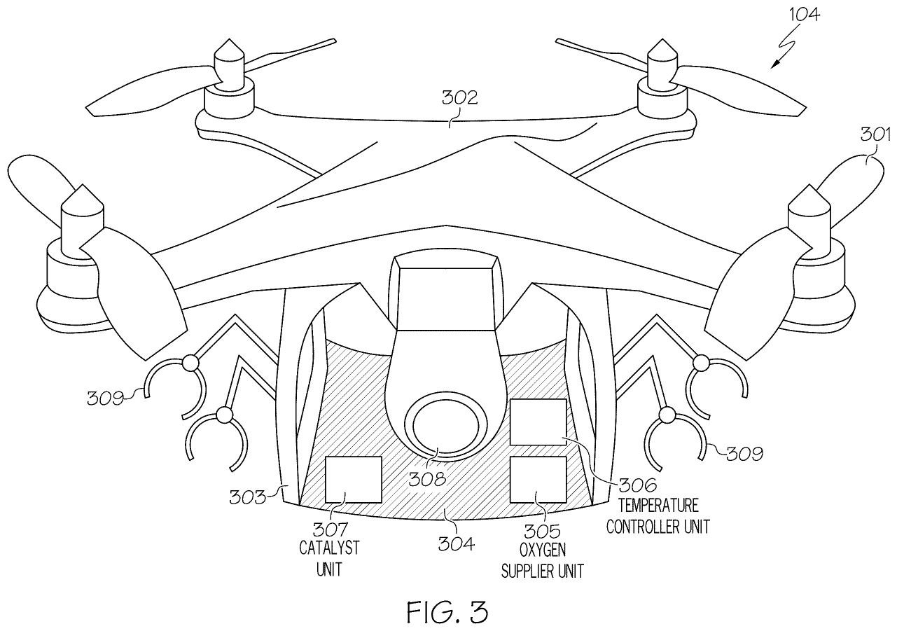

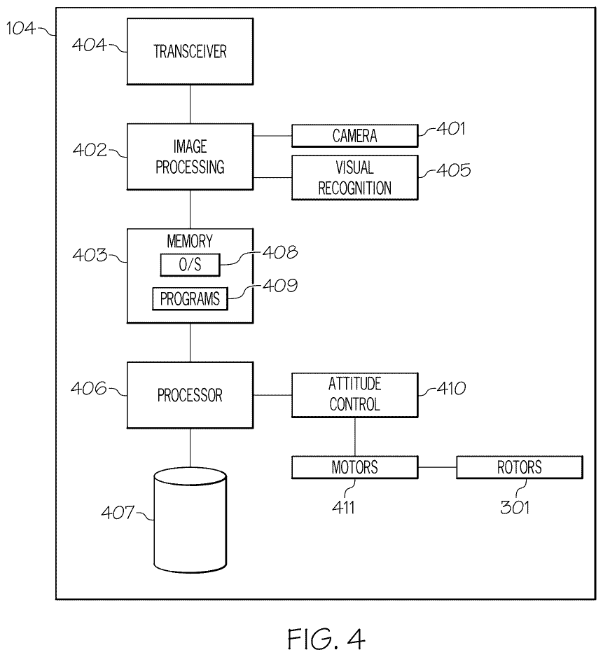

A description of a perspective view of drone 101 is provided below in connection with FIG. 3. Furthermore, a description of a hardware configuration of drone 104 is provided below in connection with FIG. 4.

As discussed above, drones 104 are connected to methane lake manager 102 via network 103. Network 103 may be, for example, a local area network, a wide area network, a wireless wide area network, a circuit-switched telephone network, a Global System for Mobile Communications (GSM) network, a Wireless Application Protocol (WAP) network, a WiFi network, an IEEE 802.11 standards network, various combinations thereof, etc. Other networks, whose descriptions are omitted here for brevity, may also be used in conjunction with system 100 of FIG. 1 without departing from the scope of the present disclosure.

Furthermore, as discussed above, system 100 includes methane leak manger 102, which is configured to manage methane leaks at a mining facility in a manner that prevents methane explosions with minimal impact on productivity.

In one embodiment, methane leak manager 102 monitors the levels of concentration of methane in the air, such as at the mining facility, using methane sensors. Upon detecting a level of concentration of methane in the air exceeding a threshold value, which may be user-designated, methane leak manager 102 creates a virtual fence around the methane sensor to identify the area where the level of concentration of methane in the air is unsafe.

Furthermore, in one embodiment, methane leak manager 102 is configured to identify mining machines 101 within and surrounding the virtual fence with a risk of setting a fire that exceeds a threshold value. As a result, in one embodiment, the work order schedule of the identified mining machines 101 are altered by having the scheduled operations of the identified mining machines 101 being performed by alternative mining machines 101. A work order schedule, as used herein, refers to a plan for tools, such as mining machines 101, that outlines when operations (e.g., drilling holes, crushing and grinding ore, transporting extracted ore, etc.) will be completed. An alternative mining machine, as used herein, refers to a mining machine (e.g., mining machine 101B) that may perform the operation originally assigned to the mining machine (e.g., mining machine 101A) with a risk of setting a fire that exceeds a threshold value.

In one embodiment, such a work order schedule for mining machines 101 that are identified as posing a risk of setting a fire is altered using a trained machine learning model, where such a machine learning model is built and trained by methane leak manager 102. In one embodiment, methane leak manager 102 trains the machine learning model to alter the work order schedule for those mining machines 101 that pose a risk of setting a fire based on a sample data set, which may include information, such as, but not limited to, operations of the mining machines (e.g., mining machine 101A) that pose a risk of setting a fire that were performed by alternative mining machines (e.g., mining machine 101B) based on functionality, the current work order schedule of the alternative mining machines (e.g., mining machine 101B), the risks of setting a fire with various levels of concentration of methane in the air by mining machines 101, the business criticality (the importance of a system or process to a company's core operations) of mining machines 101, the functionality of mining machines 101, etc. Such information is stored in a database 105 connected to methane leak manager 102.

In one embodiment, methane leak manager 102 is configured to develop a methane removal strategy for removing methane within the virtual fence, such as based, at least in part, on the cost of deploying drones 104 for removing the methane within the virtual fence. Examples of methane removal strategies include, but are not limited to, deploying drones 104 to remove the methane within the fence using diffusion or adsorption, implementing a ventilation-based system, etc.

A further discussion regarding these and other features is provided below.

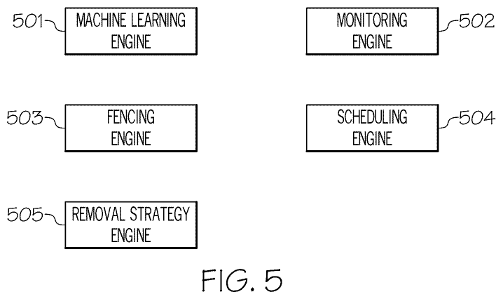

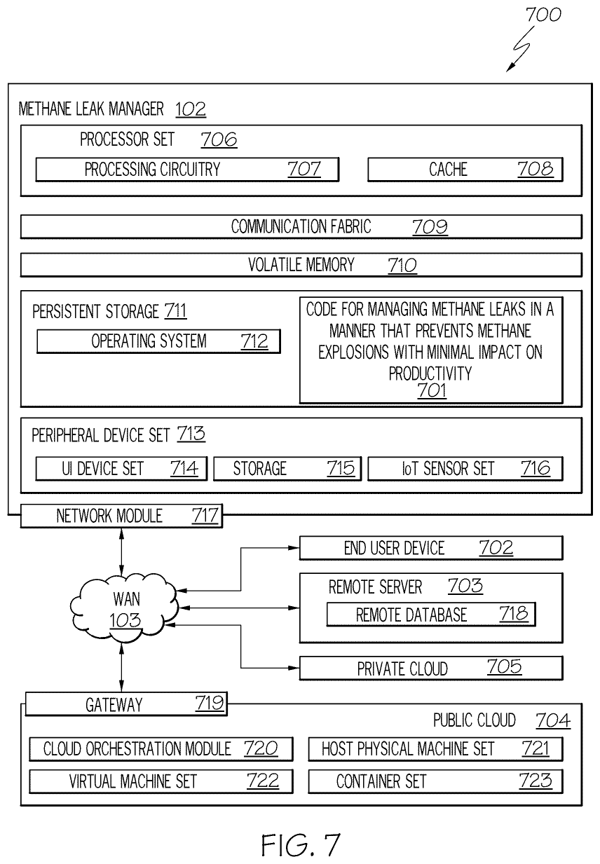

A description of the software components of methane leak manager 102 used for managing methane leaks at a mining facility in a manner that prevents methane explosions with minimal impact on productivity is provided below in connection with FIG. 5. A description of the hardware configuration of methane leak manager 102 is provided further below in connection with FIG. 7.

System 100 is not to be limited in scope to any one particular network architecture. System 100 may include any number of mining machines 101, methane leak managers 102, networks 103, drones 104, and databases 105.

Referring now to FIG. 2, FIG. 2 illustrates the internal components of mining machine 101, such as an autonomous mining machine, in accordance with an embodiment of the present disclosure.

As shown in FIG. 2, in conjunction with FIG. 1, mining machine 101 includes, but is not limited to, perception and planning system 201, vehicle control system 202, wireless communication system 203, user interface system 204, and sensor system 205. Mining machine 101 may further include certain common components included in ordinary vehicles, such as, an engine, wheels, steering wheel, transmission, etc., which may be controlled by vehicle control system 202 and/or perception and planning system 201 using a variety of communication signals and/or commands, such as, for example, acceleration signals or commands, deceleration signals or commands, steering signals or commands, braking signals or commands, etc.

Components 201-205 may be communicatively coupled to each other via an interconnect, a bus, a network, or a combination thereof. For example, components 201-205 may be communicatively coupled to each other via a controller area network (CAN) bus. A CAN bus is a vehicle bus standard designed to allow microcontrollers and devices to communicate with each other in applications without a host computer.

In one embodiment, sensor system 205 includes, but it is not limited to, one or more sensors 206, such as Internet of Things (IoT) sensors, one or more cameras 207, global positioning system (GPS) unit 208, inertial measurement unit (IMU) 209, radar unit 210, and a light detection and range (LiDAR) unit 211. GPS unit 208 may include a transceiver operable to provide information regarding the position of mining machine 101. IMU 209 may sense position and orientation changes of mining machine 101 based on inertial acceleration. Radar unit 210 may represent a system that utilizes radio signals to sense objects within the local environment of mining machine 101. In one embodiment, in addition to sensing objects, radar unit 210 may additionally sense the speed and/or heading of the objects. LiDAR unit 211 may sense objects in the environment in which mining machine 101 is located using lasers. LiDAR unit 211 could include one or more laser sources, a laser scanner, and one or more detectors, among other system components. Cameras 207 may include one or more devices to capture images of the environment surrounding mining machine 101. Cameras 207 may be still cameras and/or video cameras. A camera may be mechanically movable, for example, by mounting the camera on a rotating and/or tilting a platform.

Sensor system 205 may further include other sensors, such as, a sonar sensor, an infrared sensor, a steering sensor, a throttle sensor, a braking sensor, and an audio sensor (e.g., microphone). An audio sensor may be configured to capture sound from the environment surrounding the autonomous vehicle. A steering sensor may be configured to sense the steering angle of a steering wheel, wheels of the vehicle, or a combination thereof. A throttle sensor and a braking sensor sense the throttle position and braking position of the vehicle, respectively. In some situations, a throttle sensor and a braking sensor may be integrated as an integrated throttle/braking sensor.

In one embodiment, vehicle control system 202 includes, but are not limited to, steering unit 212, throttle unit 213 (also referred to as an acceleration unit), and braking unit 214. Steering unit 212 is to adjust the direction or heading of the vehicle. Throttle unit 213 is to control the speed of the motor or engine that in turn controls the speed and acceleration of the vehicle. Braking unit 214 is to decelerate the vehicle by providing friction to slow the wheels or tires of the vehicle.

Furthermore, in one embodiment, wireless communication system 203 is to allow communication between mining machine 101 and external systems, such as methane leak manager 102. For example, wireless communication system 203 can wirelessly communicate with one or more devices directly or via a communication network, such as methane leak manager 102 over network 103. Wireless communication system 203 can use any cellular communication network or a wireless local area network (WLAN) (e.g., using WiFi to communicate with another component or system). In one embodiment, wireless communication system 203 communicates directly with a device (e.g., a speaker within mining machine 101), for example, using an infrared link, Bluetooth, etc.

In one embodiment, user interface system 204 is part of the peripheral devices implemented within mining machine 101 including, for example, a keyboard, a touch screen display device, a microphone, a speaker, etc.

Some or all of the functions of mining machine 101 may be controlled or managed by perception and planning system 201, especially when operating in an autonomous driving mode. Perception and planning system 201 includes the necessary hardware (e.g., processor(s), memory, storage) and software (e.g., operating system, planning and routing programs) to receive information from sensor system 205, vehicle control system 202, wireless communication system 203, and/or user interface system 204, process the received information, plan a route or path from a starting point to a destination point, and then drive mining machine 101 based on the planning and control information. Alternatively, perception and planning system 201 may be integrated with vehicle control system 202.

For example, methane leak manager 102 specifies a starting location and a destination of a trip, for example, via a user interface. Perception and planning system 201 obtains the trip related data. For example, perception and planning system 201 may obtain location and route information from methane leak manager 102. For instance, methane leak manager 102 provides location and map services. Alternatively, such location and map services information may be cached locally in a persistent storage device of perception and planning system 201.

While mining machine 101 is moving along the route, perception and planning system 201 may also obtain real-time traffic information from methane leak manager 102, which obtained such information from a traffic information system or server (TIS). Based on the real-time traffic information, location information, as well as real-time local environment data detected or sensed by sensor system 205 (e.g., obstacles, objects, nearby vehicles), methane leak manager 102 and/or perception and planning system 201 can plan an optimal route, where perception and planning system 201 drives mining machine 101, for example, via vehicle control system 202, according to the planned route to reach the specified destination safely and efficiently.

In one embodiment, perception and planning system 201 includes a memory 215 for storing a localization module 216, perception module 217, prediction module 218, decision module 219, planning module 220, control module 221, routing module 222, and controller interface module 223.

In one embodiment, such modules (modules 216-223) are installed in persistent storage device 224, loaded into memory 215, and executed by one or more processors (not shown). It is noted that some or all of these modules may be communicatively coupled to or integrated with some or all modules of vehicle control system 202 of FIG. 2. Some of modules 216-223 may be integrated together as an integrated module.

In one embodiment, localization module 216 determines a current location of mining machine 101 (e.g., leveraging GPS unit 208) and manages any data related to a trip or route of mining machine 101. Localization module 216 (also referred to as a map and route module) manages any data related to a trip or route of mining machine 101. Localization module 216 communicates with other components of mining machine 101, such as map and route information 225, to obtain the trip related data. For example, localization module 216 may obtain location and route information from methane leak manager 102. Methane leak manager 102 provides location and map services, which may be cached as part of map and route information 225. While mining machine 101 is moving along the route, localization module 216 may also obtain real-time traffic information from methane leak manager 102 and/or a traffic information system or server.

Based on the sensor data provided by sensor system 205 and localization information obtained by localization module 216, a perception of the surrounding environment is determined by perception module 217. The perception information may represent what an ordinary driver would perceive surrounding a vehicle in which the driver is driving. The perception can include a relative position of another vehicle, a building, mounds of dirt, etc., for example, in a form of an object.

Perception module 217 may include a computer vision system or functionalities of a computer vision system to process and analyze images captured by one or more cameras in order to identify objects and/or features in the environment of mining machine 101. The objects can include other vehicles, obstacles, etc. The computer vision system may use an object recognition algorithm, video tracking, and other computer vision techniques. In some embodiments, the computer vision system can map an environment, track objects, and estimate the speed of objects, etc. Perception module 217 can also detect objects based on other data provided by other sensors, such as a radar and/or LiDAR.

For each of the objects, prediction module 218 predicts what the object will behave under the circumstances. The prediction is performed based on perception module 217 perceiving the driving environment at the point in time in view of a set of map and route information 225 and driving/traffic rules 226. For example, if the object is a vehicle at an opposing direction and the current driving environment includes a hole previously dug out, prediction module 218 will predict whether the vehicle will likely move straight forward or make a turn.

For each of the objects, decision module 219 makes a decision regarding how to handle the object. For example, for a particular object (e.g., another vehicle in a crossing route) as well as its metadata describing the object (e.g., a speed, direction, turning angle), decision module 219 decides how to encounter the object (e.g., overtake, yield, stop, pass). Decision module 219 may make such decisions according to a set of rules, such as traffic rules or driving rules 226, which may be stored in persistent storage device 224.

In one embodiment, methane leak manager 102 and/or routing module 222 are configured to provide one or more routes or paths from a starting point to a destination point. In one embodiment, for a given trip from a start location to a destination location, for example, received from methane leak manager 102, routing module 222 obtains map and route information 225 and determines all possible routes or paths from the starting location to reach the destination location. Routing module 222 may generate a reference line in a form of a topographic map for each of the routes it determines from the starting location to reach the destination location. A reference line refers to an ideal route or path without any interference from others, such as other vehicles, obstacles, etc. That is, if there is no other vehicle, pedestrians, or obstacles on the road, an autonomous vehicle should exactly or closely follow the reference line. The topographic maps are then provided to decision module 219 and/or planning module 220. Decision module 219 and/or planning module 220 examine all of the possible routes to select and modify one of the most optimal routes in view of other data provided by other modules, such as traffic conditions from localization module 216, driving environment perceived by perception module 217, and traffic conditions predicted by prediction module 218. The actual path or route for mining machine 101 may be close to or different from the reference line provided by methane leak manager 102 and/or routing module 222 dependent upon the specific driving environment at the point in time.

Based on a decision for each of the objects perceived, planning module 220 plans a path or route for mining machine 101 as well as driving parameters (e.g., distance, speed, and/or turning angle) using a reference line provided by routing module 222 as a basis. Alternatively, such a path or route for mining machine 101 as well as driving parameters (e.g., distance, speed, and/or turning angle) are received from methane leak manager 102.

In one embodiment, for a given object, decision module 219 decides what to do with the object, while planning module 220 determines how to do it. For example, for a given object, decision module 219 may decide to pass the object, while planning module 220 may determine whether to pass on the left side or right side of the object. Planning and control data is generated by planning module 220 including information describing how mining machine 101 would move in a next moving cycle (e.g., next route/path segment). For example, the planning and control data may instruct mining machine 101 to move to the left 10 meters at a speed of 5 miles per hour (mph), then move to the right 15 meters at the speed of 8 mph.

Based on the planning and control data, control module 221 controls and drives mining machine 101, by sending proper commands or signals to vehicle control system 202, according to a route or path defined by the planning and control data. The planning and control data includes sufficient information to drive the vehicle from a first point to a second point of a route or path using appropriate vehicle settings or driving parameters (e.g., throttle, braking, steering commands) at different points in time along the path or route.

In one embodiment, the planning phase is performed in a number of planning cycles, also referred to as driving cycles, such as, for example, in every time interval of 100 milliseconds (ms). For each of the planning cycles or driving cycles, one or more control commands will be issued based on the planning and control data. That is, for every 100 ms, planning module 220 plans a next route segment or path segment, for example, including a target position and the time required for mining machine 101 to reach the target position. Alternatively, planning module 220 may further specify the specific speed, direction, and/or steering angle, etc. In one embodiment, planning module 220 plans a route segment or path segment for the next predetermined period of time, such as 5 seconds. For each planning cycle, planning module 220 plans a target position for the current cycle (e.g., next 5 seconds) based on a target position planned in a previous cycle. Control module 221 then generates one or more control commands (e.g., throttle, brake, steering control commands) based on the planning and control data of the current cycle.

It is noted that decision module 219 and planning module 220 may be integrated as an integrated module. Decision module 219/planning module 220 may include a navigation system or functionalities of a navigation system to determine a driving path for mining machine 101. For example, the navigation system may determine a series of speeds and directional headings to affect movement of mining machine 101 along a path that substantially avoids perceived obstacles while generally advancing mining machine 101 along a path leading to an ultimate destination. The destination may be set according to inputs from methane leak manager 102. The navigation system may update the driving path dynamically while mining machine 101 is in operation. The navigation system can incorporate data from a GPS system and one or more maps so as to determine the driving path for mining machine 101.

In one embodiment, controller interface module 223 is configured to communicate with methane leak manager 102, and receive control commands from methane leak manager 102. When methane leak manager 102 issues commands to mining machine 101, the commands are forwarded to control module 221. Control module 221 may generate control signals to operate mining machine 101 in accordance with the commands received from methane leak manager 102.

A discussion regarding a perspective view of drone 104 and the hardware configuration of drone 104 is provided below in connection with FIGS. 3-4.

FIG. 3 illustrates a perspective view of drone 104 in accordance with an embodiment of the present disclosure.

Referring to FIG. 3, in conjunction with FIG. 1, drone 104 may be a commercially available unmanned aerial vehicle (UAV) platform (e.g., Amazon Prime® Air drones, DJI® Inspire 1 Pro, Yuneec® Tornado H920, etc.) modified to implement the features discussed herein. In one embodiment, drone 104 includes rotors 301 attached to a body 302. A lower frame 303 is located on a bottom portion of body 302 and is utilized to support a holder 304 for holding one or more oxygen supplier units 305, one or more temperature controller units 306, and one or more catalyst units 307.

Oxygen supplier unit 305 is configured to store oxygen, which is used by drone 104 to remove methane because it combines with methane to form carbon dioxide, a less potent greenhouse gas. Examples of oxygen supplier unit 305 include, but are not limited to, Rhythm P2-S3 portable concentrator, Inogen One® G4 portable concentrator, etc.

Temperature controller unit 306 is utilized by drone 104 for removing methane because it affects how the gas breaks down and the energy required for the process. Temperature controller unit 306, as used herein, is a device designed to precisely maintain a desired temperature, such as by actively adding or removing heat to reach a specific setpoint temperature within the process. Examples of temperature controller unit 306 include, but are not limited to, W1209 temperature controller module, Rigid Micro DC Aircon, etc.

Catalyst unit 307 is utilized by drone 104 for removing methane by using catalysts provided by catalyst unit 307 to accelerate the oxidation of methane by air, turning it into less harmful substances, such as carbon dioxide. Examples of catalysts include, but are not limited to, aluminosilicate minerals, palladium-based catalysts, mechanochemically prepared catalysts, etc.

Furthermore, in one embodiment, drones 104 are used to remove methane via diffusion using metal catalysts provided by catalyst unit 307, such as zeolite, copper-zinc oxide, etc., or photocatalysts, such as tin oxide, etc. Removing methane by diffusion, as used herein, refers to the process where methane gas moves from a region of high concentration (e.g., area within the virtual fence) to a region of low concentration (e.g., atmosphere) through the physical process of diffusion thereby allowing the methane to disperse and gradually dissipate into the surrounding air.

In one embodiment, drones 104 are used to remove methane via adsorption. Removing methane by adsorption, as used herein, refers to utilizing adsorbents to remove methane. Examples of such adsorbents provided by catalyst unit 307 include, but are not limited to, metal-organic frameworks, biochar, nano-size zeolites, zeolites, etc.

An example of catalyst unit 307 providing the catalysts, adsorbents, etc. discussed above, includes a catalytic methane abatement system.

In one embodiment, lower frame 303 is further configured to support landing drone 104 to rest on a flat surface and absorb impact during landing. Drone 104 further includes one or more sensors 308, such as cameras, which are used to take still photographs, video, and the like.

In one embodiment, drone 104 includes various electronic components inside body 302 and/or sensor 308 (e.g., camera), such as, without limitation, a processor, a data store, memory, a wireless interface, and the like. Also, drone 104 can include additional hardware, such as robotic arms 309 or pickup tongs or the like that allow the drone 104 to attach/detach/rearrange/move items.

Referring now to FIG. 4, FIG. 4 illustrates the hardware configuration of drone 104 in accordance with an embodiment of the present disclosure.

Referring to FIG. 4, in conjunction with FIGS. 1 and 3, drone 104 includes an onboard camera 401 (see element 308), such as for capturing an image of the area (e.g., within virtual fence) where methane is being removed by drone 104. For example, camera 401 may be connected to an image processing system 402. Image processing system 402 may compress the incoming stream of images for broadcast or retransmission. Image processing system 402 may store a processed image stream based on captured images in onboard memory 403, or transmit the image stream to a wireless device or methane leak manger 102 via a wireless transceiver 404.

In one embodiment, upon drones 104 receiving instructions from methane leak manager 102 as to the order and manner in removing methane, such as within a virtual fence, such drones 104 communicate amongst each other via wireless transceiver 404.

Drone 104 may further include a visual recognition system 405 connected to image processing system 402. Visual recognition system 405 may include one or more processors configured to analyze captured images, such as the captured image of the area within the virtual fence where methane is to be removed.

Furthermore, drone 104 includes a processor 406 connected to memory 403 for executing software instructions. Processor 406 may be any custom made or commercially available processor, a central processing unit, an auxiliary processor among several processors, a semiconductor-based microprocessor (in the form of a microchip or chip set) or generally any device for executing software instructions.

Additionally, drone 104 includes a data store 407 connected to processor 406 to store data. Data store 407 may include any volatile memory elements (e.g., random access memory (RAM, such as DRAM, SRAM, SDRAM, and the like), nonvolatile memory elements (e.g., ROM, hard drive, tape, CDROM, and the like), and combinations thereof). Moreover, data store 407 may incorporate electronic, magnetic, optical, and/or other types of storage media.

Memory 403 may include any volatile memory elements (e.g., random access memory (RAM, such as DRAM, SRAM, SDRAM, etc.), nonvolatile memory elements (e.g., ROM, hard drive, etc.), and combinations thereof). Moreover, memory 403 may incorporate electronic, magnetic, optical, and/or other types of storage media. It is noted that memory 403 may have a distributed architecture, where various components are situated remotely from one another but can be accessed by processor 406. The software in memory 403 may include one or more software programs, each of which includes an ordered listing of executable instructions for implementing logical functions. In the example of FIG. 4, the software in memory 403 includes a suitable operating system (O/S) 408 and programs 409. Operating system 408 essentially controls the execution of other computer programs and provides scheduling, input-output control, file and data management, memory management, and communication control and related services. Program 409 may include various applications, add-ons, etc. configured to assist methane leak manger 102 in directing drones 104 to manage methane leaks as discussed herein.

Additionally, drone 104 further includes an attitude control system 410 connected to processor 406. In one embodiment, attitude control system 410 executes any necessary changes in heading, position, or velocity by varying the rotational speed of one or more rotors 301 of drone 104.

In one embodiment, the rotor speeds of each rotor 301 may be directly controlled by one or more motors 411 connected to attitude control system 410. For example, a multi-rotor drone 104 may include four, six, eight, or any other appropriate number of rotors 301. Adjusting the speed of one or more rotors 301 may control the height of drone 104, rotate drone 104 along multiple rotational axes or degrees of freedom, or propel drone 104 in any desired direction at variable speeds.

These and other features will be discussed in greater detail further below.

Referring now to FIG. 5, FIG. 5 is a diagram of the software components of methane leak manager 102 used to manage methane leaks at a mining facility in a manner that prevents methane explosions with minimal impact on productivity.

As shown in FIG. 5, methane leak manager 102 includes machine learning engine 501, which builds and trains a machine learning model to make decisions, such as altering the work order schedule of mining machines 101 identified as posing a risk of setting a fire in an area (e.g., within the virtual fence, which is discussed further below) where the level of concentration of methane exceeds a threshold value, which may correspond to an unsafe level of concentration of methane, which may be user-designated. In one embodiment, the work order schedule for such a mining machine 101 that poses a risk of setting a fire is altered by having the previously scheduled operations of such a mining machine 101 being performed by an alternative mining machine(s) 101.

As discussed above, a work order schedule, as used herein, refers to a plan for tools, such as mining machines 101, that outlines when operations (e.g., drilling holes, crushing and grinding ore, transporting extracted ore, etc.) will be completed. An alternative mining machine 101, as used herein, refers to a mining machine (e.g., mining machine 101B) that may perform the operation originally assigned to the mining machine (e.g., mining machine 101A) that has a risk of setting a fire that exceeds a threshold value.

In one embodiment, machine learning engine 501 trains the machine learning model to alter the work order schedule for those mining machines 101 that pose a risk of setting a fire based on a sample data set, which may include information, such as, but not limited to, operations of the mining machines (e.g., mining machine 101A) that pose a risk of setting a fire that were performed by alternative mining machines (e.g., mining machine 101B) based on functionality, the work order schedule of mining machines 101 (including both the mining machines 101 identified as posing a risk of setting a fire and those available alternative mining machines 101), the risks of setting a fire with various levels of concentration of methane in the air by mining machines 101 (including both the mining machines 101 identified as posing a risk of setting a fire and those available alternative mining machines 101), the business criticality (the importance of a system or process to a company's core operations) of mining machines 101 (including both the mining machines 101 identified as posing a risk of setting a fire and those available alternative mining machines 101), the functionality of mining machines 101 (including both the mining machines 101 identified as posing a risk of setting a fire and those available alternative mining machines 101), etc. Such information (sample data set) is stored in database 105 connected to methane leak manager 102. In one embodiment, such a sample data set is populated by an expert.

Furthermore, in one embodiment, the sample data set discussed above is referred to herein as the “training data,” which is used by a machine learning algorithm to make predictions or decisions, such as altering the work order schedules of those mining machines 101 identified as posing a risk of setting a fire, such as in the area of the visual fence, where the concentration level of methane exceeds a safe level. Such work order schedules may be altered by assigning alternative mining machine(s) 101 to perform the operations previously required to be performed by those mining machines 101 identified as posing a risk of setting a fire, such as in the area of the visual fence. Such decisions by the trained machine learning model are based on the available alternative mining machines 101, the current work order schedule of the alternative mining machines 101, the functionality of mining machines 101 (including both the mining machines 101 identified as posing a risk of setting a fire and those available alternative mining machines 101), the risk of the available alternative mining machines 101 setting a fire, the business criticality of mining machines 101 (including both the mining machines 101 identified as posing a risk of setting a fire and those available alternative mining machines 101), etc. The algorithm iteratively makes predictions on the training data until the predictions achieve the desired accuracy as determined by an expert. Examples of such learning algorithms include nearest neighbor, Naïve Bayes, decision trees, linear regression, support vector machines, and neural networks.

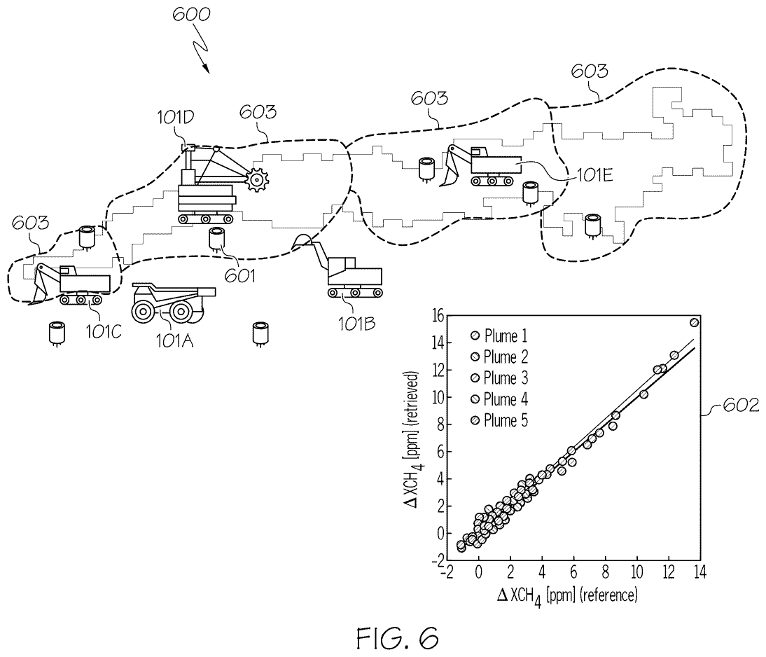

Additionally, methane leak manager 102 includes monitoring engine 502 configured to monitor levels of concentration of methane in the air using methane sensors as illustrated in FIG. 6. A methane sensor, as used herein, is a device designed to detect and measure the concentration of methane gas in the air. Furthermore, in one embodiment, such methane sensors may be configured to issue an alert when concentrations exceed a threshold value, which may be user-designated, which may correspond to an unsafe limit.

Referring to FIG. 6, FIG. 6 illustrates monitoring for methane leaks, such as at a mining facility, in accordance with an embodiment of the present disclosure.

As shown in FIG. 6, methane leaks at a mining facility 600 are monitored using methane sensors 601 placed at various locations throughout mining facility 600. Examples of methane sensors 601 include, but are not limited to, MM256, MM263, MM264, Guardian NG by Guardian Monitoring®, Gascard NG, etc.

In one embodiment, monitoring engine 502 monitors the levels of concentration of methane in the air using such methane sensors 601.

In one embodiment, monitoring engine 502 monitors for plumes of methane as shown in insert 602. A plume of methane, as used herein, refers to a large, concentrated mass of methane gas. In one embodiment, monitoring engine 502 detects plumes of methane via the use of hyperspectral satellites. In another embodiment, monitoring engine 502 detects plumes of methane via the use of sonar.

In situations in which a monitored level of concentration of methane in the air exceeds a threshold value, which may be user-designated, which may correspond to an unsafe limit, fencing engine 503 of methane leak manager 102 creates a virtual fence 603 around methane sensor 601 which identified a concentration level of methane in the air that exceeded a threshold value. Such a virtual fence 603 identifies an area where the level of concentration of methane in the air exceeds a threshold value. As discussed above, virtual fence 603, as used herein, refers to an invisible border, which marks the area where the level of concentration of methane in the air exceeds the threshold value.

In one embodiment, fencing engine 503 creates virtual fence 603 based on the concentration level of methane detected by methane sensor 601. For example, the higher the level of concentration of methane above the threshold value, the wider the dimensions of virtual fence 603. In one embodiment, fencing engine 503 may also utilize data, such as the detected plumes of methane, to determine the dimensions of virtual fence 603. In one embodiment, such dimensions of virtual fence 603 cover the area where the concentration level of methane is deemed to be unsafe.

In one embodiment, fencing engine 503 utilizes a trained machine learning model to determine the dimension of virtual fence 603. In one embodiment, machine learning engine 501 builds and trains a machine learning model to determine the dimensions of virtual fence 603 based on the concentration level of methane detected by methane sensor 601 and/or the detected plumes of methane. In one embodiment, such a machine learning model is trained by machine learning engine 501 using a sample data set that includes dimensions of virtual fences 603 in association with concentration levels of methane detected by methane sensor 601 and/or the detected plumes of methane. In one embodiment, such a sample data set resides within a storage device, such as a storage device within methane leak manger 102, or within database 105. In one embodiment, such a sample data set is populated by an expert.

Furthermore, in one embodiment, the sample data set discussed above is referred to herein as the “training data,” which is used by a machine learning algorithm to make predictions or decisions, such as detecting or predicting the dimensions of virtual fences 603 in association with concentration levels of methane detected by methane sensor 601 and/or the detected plumes of methane. The algorithm iteratively makes predictions on the training data until the predictions achieve the desired accuracy as determined by an expert. Examples of such learning algorithms include nearest neighbor, Naïve Bayes, decision trees, linear regression, support vector machines, and neural networks.

Returning to FIG. 5, in conjunction with FIGS. 1-2 and 6, in one embodiment, methane leak manger 102 further includes scheduling engine 504 configured to identify mining machines 101 within and surrounding virtual fence 603 with a risk of setting a fire that exceeds a threshold value, which may be user-designated.

In one embodiment, scheduling engine 504 identifies mining machines 101 within and surrounding virtual fence 603 with a risk of setting a fire that exceeds a threshold value, which may be user-designated, based on identifying mining machines 101 in such an area and then determining the risk of such mining machines 101 setting a fire based on the concentration level of methane detected by methane sensor 601, which triggered the formation of virtual fence 603.

In one embodiment, the location of mining machines 101 in mining facility 600 is determined based on the current location of mining machine 101 obtained from localization module 216 leveraging GPS unit 208 via the use of wireless communication system 203. In one embodiment, when localization module 216 provides the current location of mining machine 101 to methane leak manager 102, localization module 216 also provides the particular type of mining machine 101 (e.g., excavator, a crusher, a wheel loader, a blasthole drill, a bucket-wheel excavator, a dozer, a dragline excavator, a grader, a highwall miner, a mining truck, a continuous miner, etc.) to methane leak manager 102. In one embodiment, each mining machine 101 is associated with an identifier, which is appended to the current location of mining machine 101, which is transmitted to methane leak manager 102 via wireless communication system 203.

In one embodiment, the risk of mining machines 101 setting a fire based on the concentration level of methane detected by methane sensor 601, which triggered the formation of virtual fence 603, is obtained by scheduling engine 504 by accessing a data structure (e.g., table), which stores a listing of risk values associated with concentration levels of methane for various types of mining machines 101 (e.g., excavator, a crusher, a wheel loader, a blasthole drill, a bucket-wheel excavator, a dozer, a dragline excavator, a grader, a highwall miner, a mining truck, a continuous miner, etc.). As a result, based on the type of mining machine 101 and the reading (concentration level of methane) from methane sensor 601, which triggered the formation of virtual fence 603, scheduling engine 504 determines the risk value associated with mining machine 101 located within or surrounding virtual fence 603 based on accessing such a data structure. In one embodiment, such a data structure resides within the storage device of methane leak manager 102. In one embodiment, such a data structure resides within database 105. In one embodiment, such a data structure is populated by an expert.

Furthermore, in one embodiment, scheduling engine 504 alters the work order schedule for such mining machines 101 within and surrounding virtual fence 603 that were identified with a risk of setting a fire that exceeds a threshold value using the trained machine learning model. In one embodiment, such a work order schedule is altered by having the previously scheduled operations of the identified mining machines 101 (those identified as posing a risk of starting a fire) being performed by alternative mining machines 101 as determined by the trained machine learning model as discussed above. For example, mining machines 101 that were identified as posing a risk of starting a fire that were previously deployed to operate within and/or surrounding virtual fence 603 are now redeployed to operate or be stationed at a different location of mining facility 600 as illustrated in FIG. 6.

Referring to FIG. 6, the work order schedule of mining machines 101A, 101B are altered such that such mining machines 101A, 101B are no longer operating within virtual fence 603. Instead, alternative mining machines 101C, 101D, 101E have been deployed to perform the operations (e.g., crushing and grinding ore, transporting extracted ore) originally assigned to mining machines 101A, 101B. In one embodiment, such a deployment involves altering the work order schedules of the alternative mining machines 101. In this manner, there is a minimal loss of productivity while enabling the methane leak detected by methane sensor 601 to be addressed as discussed further below in connection with the discussion of the methane removal strategy.

As discussed above, in one embodiment, such work order schedules for mining machines 101 that are identified as posing a risk of setting a fire as well as the work order schedules for the alternative mining machines 101 are altered using a trained machine learning model, where such a machine learning model is built and trained by machine learning engine 501. In one embodiment, machine learning engine 501 trains the machine learning model to alter the work order schedule for those mining machines 101 that pose a risk of setting a fire as well as for those alternative mining machines 101 that perform the operations originally assigned to such mining machines 101 based on a sample data set, which may include information, such as, but not limited to, operations of the mining machines (e.g., mining machine 101A) that pose a risk of setting a fire that were performed by alternative mining machines (e.g., mining machine 101B) based on functionality, the work order schedule of mining machines 101 (including both the mining machines 101 identified as posing a risk of setting a fire and those available alternative mining machines 101), the risks of setting a fire with various levels of concentration of methane in the air by mining machines 101 (including both the mining machines 101 identified as posing a risk of setting a fire and those available alternative mining machines 101), the business criticality of mining machines 101 (including both the mining machines 101 identified as posing a risk of setting a fire and those available alternative mining machines 101), the functionality of mining machines 101 (including both the mining machines 101 identified as posing a risk of setting a fire and those available alternative mining machines 101), etc. In one embodiment, such information is stored in a database 105 connected to methane leak manager 102.

In one embodiment, the reassignment of the operations from the mining machines 101 deemed to be at risk of starting a fire to the alternative mining machines 101 is performed by scheduling engine 504 by instructing mining machines 101 deemed to be at risk of starting a fire to relocate to a new position outside virtual fence 603, such as via controller interface module 223, which forwards such commands to control module 221. Control module 221 may then generate control signals to operate mining machine 101 in accordance with the commands received from scheduling engine 504.

Similarly, scheduling engine 504 instructs the alternative mining machines 101 to perform the operations (e.g., crushing and grinding ore, transporting extracted ore) at a designated location (e.g., within virtual fence 603), which were originally assigned to mining machines 101 deemed to be at risk of starting a fire, such as via controller interface module 223, which forwards such commands to control module 221. Control module 221 may then generate control signals to operate the alternative mining machines 101 in accordance with the commands received from scheduling engine 504.

Returning to FIG. 5, in conjunction with FIGS. 1 and 6, in one embodiment, methane leak manager 102 includes removal strategy engine 505 configured to develop a methane removal strategy for removing methane within virtual fence 603 based, at least in part, on the cost of deploying drones 104 (drones 104 with oxygen, temperature, and catalyst units 305, 306, 307, respectively) for removing the methane within virtual fence 603. Examples of methane removal strategies include, but are not limited to, deploying drones 104 to remove the methane within virtual fence 603 using diffusion or adsorption, implementing a ventilation-based system (e.g., regenerative thermal oxidation (RTO) system manufactured by Epcon® Industrial Systems, LP), etc.

In one embodiment, removal strategy engine 505 performs a cost-benefit analysis to identify the optimal methane removal strategy for removing methane within virtual fence 603. In one embodiment, such a cost-benefit analysis considers the energy consumption due to the deployment of drones 104 to remove the methane within virtual fence 603, and the risk assessment of starting a fire by mining machines 101 located within and surrounding virtual fence 603, as shown in the following formula:

$$J = Max . {\sum }_{j = 1}^{N} \{ {u}_{j} ( {w}_{d r o n e} \cdot {E}_{d r o n e , j} - {w}_{d r o n e} \cdot {C}_{d r o n e , j} ) + {v}_{j} ( {w}_{vent} \cdot {E}_{v e n t , j} ) \}$$

where t_{drone,j}+t_{vent,j}≤t_{critical,j} and uⱼ+vⱼ≤1,
J is the cost function,
uⱼ is the binary decision variable for drone based methane removal,
vⱼ is the binary decision variable for ventilation based removal,
E_{drone,j} is the potential amount of methane diffusion at pixel ‘j,’ which is a function of methane intensity, location, required amount of oxygen and temperature and conversion efficiency of the catalyst,
E_{vent,j} is the potential amount of methane diffusion at pixel ‘j,’ which is a function of the type of ventilation,
C_{drone,j} is the cost of deploying drones with optimal oxygen, temperature and catalyst units for the hydrocarbon conversion process at pixel ‘j,’
w_{drone}, wᵥₑₙₜ is the weighing cost coefficient for hydrocarbon fuel and CO₂ conversion, respectively, as a function of greenhouse potential and carbon credits,
t_{drone,j}, t_{vent,j} is the time taken for methane diffusion by drone and ventilation-based system, respectively,
t_{critical,j} is the critical time available for methane diffusion at pixel ‘j’ considering the mining machine fire assessment,
w_{drone}·E_{drone,j} is the drone based methane conversion process,
w_{drone}·C_{drone,j} is the cost of deploying drones for the conversion process, and
wᵥₑₙₜ·E_{vent,j} is the ventilation based process.

In one embodiment, if removal strategy engine 505 determines to deploy drones 104 to remove the methane within virtual fence 603 via diffusion, then removal strategy engine 505 determines the optimal set of drones 104 to be deployed with an optimal number of methane catalyst units 307, temperature controller units 306, and oxygen supplier units 305 to minimize the time to diffuse the methane hotspots within virtual fence(s) 603 as shown in the following formula:

$$J = Min ( \frac{C i n {t}_{1}^{t}}{{C}_{1}} + \frac{C i n {t}_{2}^{t}}{{C}_{2}} + \frac{C i n {t}_{3}^{t}}{{C}_{3}} + … + \frac{C i n {t}_{n}^{t}}{{C}_{n}} )$$

where Cint is the methane intensity at hotspot (virtual fence 603) 1 at time t,
C₁ is the methane conversion/diffusion rate (either hydrocarbon fuel or CO₂) at hotspot (virtual fence 603) 1 at time t,

$${C}_{1} = f ( x 1 \cdot MCU , x 2 \cdot OSU , x 3 \cdot TCU ) ,$$

MCU is the methane catalyst unit 307,
OSU is the oxygen supplier unit 305,
TCU is the temperature controlling unit 306, and
x1, x2, and x3 are the number of selected units.

In one embodiment, the constraints for the above-described formula are the following:

$$\frac{{Cint}_{1}^{t}}{{C}_{1}} < t 1 , \frac{{Cint}_{2}^{t}}{{C}_{2}} < t 2 , … , \frac{{Cint}_{n}^{t}}{{C}_{n}} < t n$$

In one embodiment, removal strategy engine 505 optimizes the control parameters for drone-based methane removal. In one embodiment, since the control of temperature and oxygen supply needs to be very precise and subject to small changes, the temperature and oxygen are chosen in a matter that it is optimal for the next “K” steps as shown in the following formula:

$$J = {\sum }_{i = 1}^{N} {{w}_{{H}_{i}} ( {H}_{i}^{r} - {H}_{i}^{m} )}^{2} + {\sum }_{i = 1}^{N} {{w}_{{L}_{i}} ( {L}_{i}^{r} - {L}_{i}^{m} )}^{2} + {\sum }_{i = 1}^{N} {{w}_{u {H}_{i}} ( Δ u )}^{2} + {\sum }_{i = 1}^{N} {{w}_{v {L}_{i}} ( Δ v )}^{2} \\ subject to {H}^{min} \leq {H}_{i}^{m} \leq {H}^{max} and {L}^{min} \leq {L}_{i}^{m} \leq {L}^{max}$$

where J is the cost function over the receding horizon,
Hᵢ^{r} is the required temperature for the instant ‘i’ from the knowledge base,
Hᵢ^{m} is the measured temperature for the instant ‘i,’
L is the required oxygen for the instant ‘i’ from the knowledge base,
Lᵢ^{t} is the measured oxygen for the instant ‘i,’
u, v are the temperature and oxygen controller variables, respectively,
w_{H}_{ᵢ}, w_{L}_{ᵢ} are the weighting coefficients for temperature and oxygen, respectively,
w_{uH}_{ᵢ}, w_{uL}_{ᵢ} are the penalizing coefficients for large changes in the temperature and supplied oxygen, respectively,
H^{max}, L^{max} are the maximum limits for temperature and oxygen, respectively, and
H^{min}, L^{min} are the minimum limits for temperature and oxygen, respectively.

In one embodiment, upon identifying the appropriate methane removal strategy, removal strategy engine 505 implements the appropriate methane removal strategy for removing the methane within virtual fence 603.

Upon removing the methane from virtual fence 603 in such a manner that the concentration level of methane within virtual fence 603 has been reduced to a safe level (i.e., the level of concentration of methane in the air within virtual fence 603 is less than the threshold value), scheduling engine 504 alters the work order schedule of mining machines 101, whose operations were reassigned to the alternative mining machines 101, such as to perform the operations as originally assigned to mining machines 101 prior to the detection of an unsafe level of methane to the extent that such operations were not completed by the alternative mining machines 101.

In this manner, methane leaks in mining operations are managed in a manner that prevents methane explosions with minimal impact on productivity.

A further description of these and other features is provided below in connection with the discussion of the method for managing methane leaks in a manner that prevents methane explosions with minimal impact on productivity.

Prior to the discussion of the method for managing methane leaks in a manner that prevents methane explosions with minimal impact on productivity, a description of the hardware configuration of methane leak manager 102 (FIG. 1) is provided below in connection with FIG. 7.

Referring now to FIG. 7, in conjunction with FIG. 1, FIG. 7 illustrates an embodiment of the present disclosure of the hardware configuration of methane leak manager 102 which is representative of a hardware environment for practicing the present disclosure.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 700 contains an example of an environment for the execution of at least some of the computer code which is stored in block 701 involved in performing the disclosed methods, such as managing methane leaks in a manner that prevents methane explosions with minimal impact on productivity. In addition to block 701, computing environment 700 includes, for example, methane leak manager 102, network 103, such as a wide area network (WAN), end user device (EUD) 702, remote server 703, public cloud 704, and private cloud 705. In this embodiment, methane leak manager 102 includes processor set 706 (including processing circuitry 707 and cache 708), communication fabric 709, volatile memory 710, persistent storage 711 (including operating system 712 and block 701, as identified above), peripheral device set 713 (including user interface (UI) device set 714, storage 715, and Internet of Things (IoT) sensor set 716), and network module 717. Remote server 703 includes remote database 718. Public cloud 704 includes gateway 719, cloud orchestration module 720, host physical machine set 721, virtual machine set 722, and container set 723.

Methane leak manager 102 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 718. As is well understood in the art of computer technology, and depending upon the technology, performance of a computer-implemented method may be distributed among multiple computers and/or between multiple locations. On the other hand, in this presentation of computing environment 700, detailed discussion is focused on a single computer, specifically methane leak manager 102, to keep the presentation as simple as possible. Methane leak manager 102 may be located in a cloud, even though it is not shown in a cloud in FIG. 7. On the other hand, methane leak manager 102 is not required to be in a cloud except to any extent as may be affirmatively indicated.

Processor set 706 includes one, or more, computer processors of any type now known or to be developed in the future. Processing circuitry 707 may be distributed over multiple packages, for example, multiple, coordinated integrated circuit chips. Processing circuitry 707 may implement multiple processor threads and/or multiple processor cores. Cache 708 is memory that is located in the processor chip package(s) and is typically used for data or code that should be available for rapid access by the threads or cores running on processor set 706. Cache memories are typically organized into multiple levels depending upon relative proximity to the processing circuitry. Alternatively, some, or all, of the cache for the processor set may be located “off chip.” In some computing environments, processor set 706 may be designed for working with qubits and performing quantum computing.

Computer readable program instructions are typically loaded onto methane leak manager 102 to cause a series of operational steps to be performed by processor set 706 of methane leak manager 102 and thereby effect a computer-implemented method, such that the instructions thus executed will instantiate the methods specified in flowcharts and/or narrative descriptions of computer-implemented methods included in this document (collectively referred to as “the disclosed methods”). These computer readable program instructions are stored in various types of computer readable storage media, such as cache 708 and the other storage media discussed below. The program instructions, and associated data, are accessed by processor set 706 to control and direct performance of the disclosed methods. In computing environment 700, at least some of the instructions for performing the disclosed methods may be stored in block 701 in persistent storage 711.

Communication fabric 709 is the signal conduction paths that allow the various components of methane leak manager 102 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 710 is any type of volatile memory now known or to be developed in the future. Examples include dynamic type random access memory (RAM) or static type RAM. Typically, the volatile memory is characterized by random access, but this is not required unless affirmatively indicated. In methane leak manager 102, the volatile memory 710 is located in a single package and is internal to methane leak manager 102, but, alternatively or additionally, the volatile memory may be distributed over multiple packages and/or located externally with respect to methane leak manager 102.

Persistent Storage 711 is any form of non-volatile storage for computers that is now known or to be developed in the future. The non-volatility of this storage means that the stored data is maintained regardless of whether power is being supplied to methane leak manager 102 and/or directly to persistent storage 711. Persistent storage 711 may be a read only memory (ROM), but typically at least a portion of the persistent storage allows writing of data, deletion of data and re-writing of data. Some familiar forms of persistent storage include magnetic disks and solid state storage devices. Operating system 712 may take several forms, such as various known proprietary operating systems or open source Portable Operating System Interface type operating systems that employ a kernel. The code included in block 701 typically includes at least some of the computer code involved in performing the disclosed methods.

Peripheral device set 713 includes the set of peripheral devices of methane leak manager 102. Data communication connections between the peripheral devices and the other components of methane leak manager 102 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 714 may include components such as a display screen, speaker, microphone, wearable devices (such as goggles and smart watches), keyboard, mouse, printer, touchpad, game controllers, and haptic devices. Storage 715 is external storage, such as an external hard drive, or insertable storage, such as an SD card. Storage 715 may be persistent and/or volatile. In some embodiments, storage 715 may take the form of a quantum computing storage device for storing data in the form of qubits. In embodiments where methane leak manager 102 is required to have a large amount of storage (for example, where methane leak manager 102 locally stores and manages a large database) then this storage may be provided by peripheral storage devices designed for storing very large amounts of data, such as a storage area network (SAN) that is shared by multiple, geographically distributed computers. IoT sensor set 716 is made up of sensors that can be used in Internet of Things applications. For example, one sensor may be a thermometer and another sensor may be a motion detector.

Network module 717 is the collection of computer software, hardware, and firmware that allows methane leak manager 102 to communicate with other computers through WAN 103. Network module 717 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 717 are performed on the same physical hardware device. In other embodiments (for example, embodiments that utilize software-defined networking (SDN)), the control functions and the forwarding functions of network module 717 are performed on physically separate devices, such that the control functions manage several different network hardware devices. Computer readable program instructions for performing the disclosed methods can typically be downloaded to methane leak manager 102 from an external computer or external storage device through a network adapter card or network interface included in network module 717.

WAN 103 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 702 is any computer system that is used and controlled by an end user (for example, a customer of an enterprise that operates methane leak manager 102), and may take any of the forms discussed above in connection with methane leak manager 102. EUD 702 typically receives helpful and useful data from the operations of methane leak manager 102. For example, in a hypothetical case where methane leak manager 102 is designed to provide a recommendation to an end user, this recommendation would typically be communicated from network module 717 of methane leak manager 102 through WAN 103 to EUD 702. In this way, EUD 702 can display, or otherwise present, the recommendation to an end user. In some embodiments, EUD 702 may be a client device, such as thin client, heavy client, mainframe computer, desktop computer and so on.

Remote server 703 is any computer system that serves at least some data and/or functionality to methane leak manager 102. Remote server 703 may be controlled and used by the same entity that operates methane leak manager 102. Remote server 703 represents the machine(s) that collect and store helpful and useful data for use by other computers, such as methane leak manager 102. For example, in a hypothetical case where methane leak manager 102 is designed and programmed to provide a recommendation based on historical data, then this historical data may be provided to methane leak manager 102 from remote database 718 of remote server 703.

Public cloud 704 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 704 is performed by the computer hardware and/or software of cloud orchestration module 720. The computing resources provided by public cloud 704 are typically implemented by virtual computing environments that run on various computers making up the computers of host physical machine set 721, which is the universe of physical computers in and/or available to public cloud 704. The virtual computing environments (VCEs) typically take the form of virtual machines from virtual machine set 722 and/or containers from container set 723. It is understood that these VCEs may be stored as images and may be transferred among and between the various physical machine hosts, either as images or after instantiation of the VCE. Cloud orchestration module 720 manages the transfer and storage of images, deploys new instantiations of VCEs and manages active instantiations of VCE deployments. Gateway 719 is the collection of computer software, hardware, and firmware that allows public cloud 704 to communicate through WAN 103.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 705 is similar to public cloud 704, except that the computing resources are only available for use by a single enterprise. While private cloud 705 is depicted as being in communication with WAN 103 in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 704 and private cloud 705 are both part of a larger hybrid cloud.

Block 701 further includes the software components discussed above in connection with FIGS. 5-6 to manage methane leaks in a manner that prevents methane explosions with minimal impact on productivity. In one embodiment, such components may be implemented in hardware. The functions discussed above performed by such components are not generic computer functions. As a result, methane leak manager 102 is a particular machine that is the result of implementing specific, non-generic computer functions.

In one embodiment, the functionality of such software components of methane leak manager 102, including the functionality for managing methane leaks in a manner that prevents methane explosions with minimal impact on productivity, may be embodied in an application specific integrated circuit.

As stated above, mining operations may involve the leakage of methane (CH₄), such as from coal seams and surrounding rock strata. For example, methane may be released from coal seams. When coal seams are fractured during mining, the methane trapped under pressure is released. In surface mines, drainage systems are a source of methane emissions. Furthermore, methane can seep out during the processing, storage, and transport of coal in underground mines. Additionally, methane can be released from abandoned mines. Methane is a greenhouse gas that is explosive when mixed with air so it poses a safety risk. An explosion may be set off by an electrostatic spark creating a huge increase in gas pressure. Such an electrostatic spark may be caused by the mining machines operating in the area where the concentration level of methane has reached an unsafe level. Such methane explosions can lead to mine evacuations, shutdowns, and stoppages, which can cause significant delays in production and loss of revenue. Unfortunately, there is not currently a means for managing methane leaks in mining operations in a manner that prevents methane explosions with minimal impact on productivity.

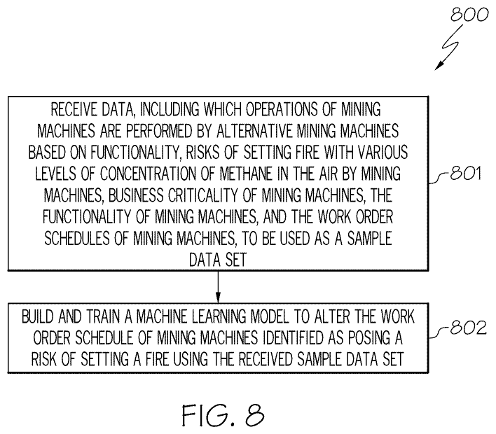

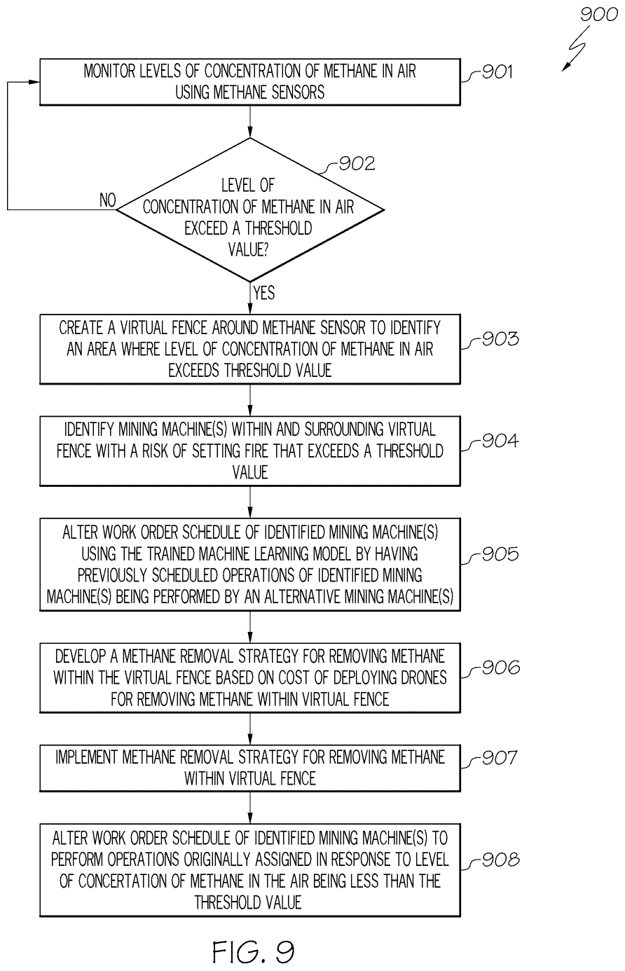

The embodiments of the present disclosure provide a means for managing methane leaks in mining operations in a manner that prevents methane explosions with minimal impact on productivity as discussed below in connection with FIGS. 8-9. FIG. 8 is a flowchart of a method for training a machine learning model for altering the work order schedules of those mining machines located within or surrounding a virtual fence with a risk of setting a fire that exceeds a threshold value. FIG. 9 is a flowchart of a method for managing methane leaks in mining operations in a manner that prevents methane explosions with minimal impact on productivity.

As stated above, FIG. 8 a flowchart of a method 800 for training a machine learning model for altering the work order schedules of those mining machines (e.g., mining machines 101 of FIG. 1) located within or surrounding a virtual fence (e.g., virtual fence 603 of FIG. 6) with a risk of setting a fire that exceeds a threshold value in accordance with an embodiment of the present disclosure.

Referring to FIG. 8, in conjunction with FIGS. 1-7, in step 801, machine learning engine 501 of methane leak manager 102 receives data, including which operations of mining machines 101 are performed by alternative mining machines 101 based on functionality, risks of setting a fire with various levels of concentration of methane in the air by mining machines 101 (including both the mining machines 101 identified as posing a risk of setting a fire and those available alternative mining machines 101), business criticality (the importance of a system or process to a company's core operations) of mining machines 101 (including both the mining machines 101 identified as posing a risk of setting a fire and those available alternative mining machines 101), the functionality of mining machines 101 (including both the mining machines 101 identified as posing a risk of setting a fire and those available alternative mining machines 101), and the work order schedules of mining machines 101 (including both the mining machines 101 identified as posing a risk of setting a fire and those available alternative mining machines 101), to be used as a sample data set.

In step 802, machine learning engine 501 of methane leak manager 102 builds and trains a machine learning model to alter the work order schedule of mining machines 101 identified as posing a risk of setting a fire using the received sample data set.

As discussed above, machine learning engine 501 builds and trains a machine learning model to make decisions, such as altering the work order schedule of mining machines 101 identified as posing a risk of setting a fire in an area (e.g., within virtual fence 603) where the level of concentration of methane exceeds a threshold value, which may correspond to an unsafe level of concentration of methane, which may be user-designated. In one embodiment, the work order schedule for such a mining machine 101 that poses a risk of setting a fire is altered by having the previously scheduled operations of such a mining machine 101 being performed by an alternative mining machine(s) 101.

As discussed above, a work order schedule, as used herein, refers to a plan for tools, such as mining machines 101, that outlines when operations (e.g., drilling holes, crushing and grinding ore, transporting extracted ore, etc.) will be completed. An alternative mining machine 101, as used herein, refers to a mining machine (e.g., mining machine 101B) that may perform the operation originally assigned to the mining machine (e.g., mining machine 101A) that has a risk of setting a fire that exceeds a threshold value.

In one embodiment, machine learning engine 501 trains the machine learning model to alter the work order schedule for those mining machines 101 that pose a risk of setting a fire based on a sample data set, which may include information, such as, but not limited to, operations of the mining machines (e.g., mining machine 101A) that pose a risk of setting a fire that were performed by alternative mining machines (e.g., mining machine 101B) based on functionality, the work order schedule of mining machines 101 (including both the mining machines 101 identified as posing a risk of setting a fire and those available alternative mining machines 101), the risks of setting a fire with various levels of concentration of methane in the air by mining machines 101 (including both the mining machines 101 identified as posing a risk of setting a fire and those available alternative mining machines 101), the business criticality (the importance of a system or process to a company's core operations) of mining machines 101 (including both the mining machines 101 identified as posing a risk of setting a fire and those available alternative mining machines 101), the functionality of mining machines 101 (including both the mining machines 101 identified as posing a risk of setting a fire and those available alternative mining machines 101), etc. Such information (sample data set) is stored in a database 105 connected to methane leak manager 102. In one embodiment, such a sample data set is populated by an expert.

Furthermore, in one embodiment, the sample data set discussed above is referred to herein as the “training data,” which is used by a machine learning algorithm to make predictions or decisions, such as altering the work order schedules of those mining machines 101 identified as posing a risk of setting a fire, such as in the area of visual fence 603, where the concentration level of methane exceeds a safe level. Such work order schedules may be altered by assigning alternative mining machine(s) 101 to perform the operations previously required to be performed by those mining machines 101 identified as posing a risk of setting a fire, such as in the area of visual fence 603. Such decisions by the trained machine learning model are based on the available alternative mining machines 101, the current work order schedule of the alternative mining machines 101, the functionality of mining machines 101 (including both the mining machines 101 identified as posing a risk of setting a fire and those available alternative mining machines 101), the risk of the available alternative mining machines 101 setting a fire, the business criticality of mining machines 101 (including both the mining machines 101 identified as posing a risk of setting a fire and those available alternative mining machines 101), etc. The algorithm iteratively makes predictions on the training data until the predictions achieve the desired accuracy as determined by an expert. Examples of such learning algorithms include nearest neighbor, Naïve Bayes, decision trees, linear regression, support vector machines, and neural networks.

Such a trained machine learning model is utilized by methane leak manger 102 to manage methane leaks in a manner that prevents methane explosions with minimal impact on productivity as discussed below in connection with FIG. 9.

FIG. 9 is a flowchart of a method 900 for managing methane leaks in mining operations in a manner that prevents methane explosions with minimal impact on productivity in accordance with an embodiment of the present disclosure.

Referring to FIG. 9, in conjunction with FIGS. 1-8, in step 901, monitoring engine 502 of methane leak manger 102 monitors levels of concentration of methane in the air using methane sensors 601 as illustrated in FIG. 6.

As discussed above, methane sensor 601, as used herein, is a device designed to detect and measure the concentration of methane gas in the air. Furthermore, in one embodiment, such methane sensors 601 may be configured to issue an alert when concentrations exceed a threshold value, which may be user-designated, which may correspond to an unsafe limit.

As shown in FIG. 6, methane leaks at a mining facility 600 are monitored using methane sensors 601 placed at various locations throughout mining facility 600. Examples of methane sensors 601 include, but are not limited to, MM256, MM263, MM264, Guardian NG by Guardian Monitoring®, Gascard NG, etc.

In one embodiment, monitoring engine 502 monitors the levels of concentration of methane in the air using such methane sensors 601.

In one embodiment, monitoring engine 502 monitors for plumes of methane as shown in insert 602. A plume of methane, as used herein, refers to a large, concentrated mass of methane gas. In one embodiment, monitoring engine 502 detects plumes of methane via the use of hyperspectral satellites. In another embodiment, monitoring engine 502 detects plumes of methane via the use of sonar.

In step 902, monitoring engine 502 of methane leak manger 102 determines whether the level of concentration of methane in the air exceeds a threshold value, which may be user-designated.

If the level of concentration of methane in the air does not exceed the threshold value, then monitoring engine 502 continues to monitor the levels of concentration of methane in the air using methane sensors 601 in step 901.

If, however, the level of concentration of methane in the air exceeds the threshold value, which may correspond to an unsafe level, then, in step 903, fencing engine 503 of methane leak manager 102 creates a virtual fence 603 around methane sensor 601 which identified a concentration level of methane in the air that exceeded a threshold value. Such a virtual fence 603 identifies an area where the level of concentration of methane in the air exceeds a threshold value. As discussed above, virtual fence 603, as used herein, refers to an invisible border, which marks the area where the level of concentration of methane in the air exceeds the threshold value.

As discussed above, in one embodiment, fencing engine 503 creates virtual fence 603 based on the concentration level of methane detected by methane sensor 601. For example, the higher the level of concentration of methane above the threshold value, the wider the dimensions of virtual fence 603. In one embodiment, fencing engine 503 may also utilize data, such as the detected plumes of methane, to determine the dimensions of virtual fence 603. In one embodiment, such dimensions of virtual fence 603 cover the area where the concentration level of methane is deemed to be unsafe.

In one embodiment, fencing engine 503 utilizes a trained machine learning model to determine the dimension of virtual fence 603. In one embodiment, machine learning engine 501 builds and trains a machine learning model to determine the dimensions of virtual fence 603 based on the concentration level of methane detected by methane sensor 601 and/or the detected plumes of methane. In one embodiment, such a machine learning model is trained by machine learning engine 501 using a sample data set that includes dimensions of virtual fences 603 in association with concentration levels of methane detected by methane sensor 601 and/or the detected plumes of methane. In one embodiment, such a sample data set resides within a storage device, such as a storage device (e.g., storage device 711, 715) within methane leak manger 102, or within database 105. In one embodiment, such a sample data set is populated by an expert.

Furthermore, in one embodiment, the sample data set discussed above is referred to herein as the “training data,” which is used by a machine learning algorithm to make predictions or decisions, such as detecting or predicting the dimensions of virtual fences 603 in association with concentration levels of methane detected by methane sensor 601 and/or the detected plumes of methane. The algorithm iteratively makes predictions on the training data until the predictions achieve the desired accuracy as determined by an expert. Examples of such learning algorithms include nearest neighbor, Naïve Bayes, decision trees, linear regression, support vector machines, and neural networks.

In step 904, scheduling engine 504 of methane leak manager 102 identifies mining machines 101 within and surrounding virtual fence 603 with a risk of setting a fire that exceeds a threshold value, which may be user-designated.

As stated above, in one embodiment, scheduling engine 504 identifies mining machines 101 within and surrounding virtual fence 603 with a risk of setting a fire that exceeds a threshold value, which may be user-designated, based on identifying mining machines 101 in such an area and then determining the risk of such mining machines 101 setting a fire based on the concentration level of methane detected by methane sensor 601, which triggered the formation of virtual fence 603.

In one embodiment, the location of mining machines 101 in mining facility 600 is determined based on the current location of mining machine 101 obtained from localization module 216 leveraging GPS unit 208 via the use of wireless communication system 203. In one embodiment, when localization module 216 provides the current location of mining machine 101 to methane leak manager 102, localization module 216 also provides the particular type of mining machine 101 (e.g., excavator, a crusher, a wheel loader, a blasthole drill, a bucket-wheel excavator, a dozer, a dragline excavator, a grader, a highwall miner, a mining truck, a continuous miner, etc.) to methane leak manager 102. In one embodiment, each mining machine 101 is associated with an identifier, which is appended to the current location of mining machine 101, which is transmitted to methane leak manager 102 via wireless communication system 203.

In one embodiment, the risk of mining machines 101 setting a fire based on the concentration level of methane detected by methane sensor 601, which triggered the formation of virtual fence 603, is obtained by scheduling engine 504 by accessing a data structure (e.g., table), which stores a listing of risk values associated with concentration levels of methane for various types of mining machines 101 (e.g., excavator, a crusher, a wheel loader, a blasthole drill, a bucket-wheel excavator, a dozer, a dragline excavator, a grader, a highwall miner, a mining truck, a continuous miner, etc.). As a result, based on the type of mining machine 101 and the reading (concentration level of methane) from methane sensor 601, which triggered the formation of virtual fence 603, scheduling engine 504 determines the risk value associated with mining machine 101 located within or surrounding virtual fence 603 based on accessing such a data structure. In one embodiment, such a data structure resides within the storage device (e.g., storage device 711, 715) of methane leak manager 102. In one embodiment, such a data structure resides within database 105. In one embodiment, such a data structure is populated by an expert.

In step 905, scheduling engine 504 of methane leak manager 102 alters the work order schedule for such mining machines 101 within and surrounding virtual fence 603 that were identified with a risk of setting a fire that exceeds a threshold value using the trained machine learning model.

As discussed above, in one embodiment, such a work order schedule is altered by having the previously scheduled operations of the identified mining machines 101 being performed by alternative mining machines 101 as determined by the trained machine learning model as discussed above. For example, mining machines 101 that were identified as posing a risk of starting a fire that were previously deployed to operate within and/or surrounding virtual fence 603 are now redeployed to operate or be stationed at a different location of mining facility 600 as illustrated in FIG. 6.

Referring to FIG. 6, the work order schedule of mining machines 101A, 101B are altered such that such mining machines 101A, 101B are no longer operating within virtual fence 603. Instead, alternative mining machines 101C, 101D, 101E have been deployed to perform the operations (e.g., crushing and grinding ore, transporting extracted ore) originally assigned to mining machines 101A, 101B. In one embodiment, such a deployment involves altering the work order schedules of the alternative mining machines 101. In this manner, there is a minimal loss of productivity while enabling the methane leak detected by methane sensor 601 to be addressed as discussed further below in connection with the discussion of the methane removal strategy.

In one embodiment, such work order schedules for mining machines 101 that are identified as posing a risk of setting a fire as well as the work order schedules for the alternative mining machines 101 are altered using a trained machine learning model, where such a machine learning model is built and trained by machine learning engine 501. In one embodiment, machine learning engine 501 trains the machine learning model to alter the work order schedule for those mining machines 101 that pose a risk of setting a fire as well as for those alternative mining machines 101 that perform the operations originally assigned to such mining machines 101 based on a sample data set, which may include information, such as, but not limited to, operations of the mining machines (e.g., mining machine 101A) that pose a risk of setting a fire that were performed by alternative mining machines (e.g., mining machine 101B) based on functionality, the work order schedule of mining machines 101 (including both the mining machines 101 identified as posing a risk of setting a fire and those available alternative mining machines 101), the risks of setting a fire with various levels of concentration of methane in the air by mining machines 101 (including both the mining machines 101 identified as posing a risk of setting a fire and those available alternative mining machines 101), the business criticality of mining machines 101 (including both the mining machines 101 identified as posing a risk of setting a fire and those available alternative mining machines 101), the functionality of mining machines 101 (including both the mining machines 101 identified as posing a risk of setting a fire and those available alternative mining machines 101), etc. In one embodiment, such information is stored in a database 105 connected to methane leak manager 102.

In one embodiment, the reassignment of the operations from the mining machines 101 deemed to be at risk of starting a fire to the alternative mining machines 101 is performed by scheduling engine 504 by instructing mining machines 101 deemed to be at risk of starting a fire to relocate to a new position outside virtual fence 603, such as via controller interface module 223, which forwards such commands to control module 221. Control module 221 may then generate control signals to operate mining machine 101 in accordance with the commands received from scheduling engine 504.

Similarly, scheduling engine 504 instructs the alternative mining machines 101 to perform the operations (e.g., crushing and grinding ore, transporting extracted ore) at a designated location (e.g., within virtual fence 603), which were originally assigned to mining machines 101 deemed to be at risk of starting a fire, such as via controller interface module 223, which forwards such commands to control module 221. Control module 221 may then generate control signals to operate the alternative mining machines 101 in accordance with the commands received from scheduling engine 504.

In step 906, removal strategy engine 505 of methane leak manger 102 develops a methane removal strategy for removing methane within virtual fence 603 based, at least in part, on the cost of deploying drones 104 (drones 104 with oxygen, temperature, and catalyst units 305, 306, 307, respectively) for removing the methane within virtual fence 603. Examples of methane removal strategies include, but are not limited to, deploying drones 104 to remove the methane within virtual fence 603 using diffusion or adsorption, implementing a ventilation-based system (e.g., regenerative thermal oxidation (RTO) system manufactured by Epcon® Industrial Systems, LP), etc.

As stated above, in one embodiment, removal strategy engine 505 performs a cost-benefit analysis to identify the optimal methane removal strategy for removing methane within virtual fence 603. In one embodiment, such a cost-benefit analysis considers the energy consumption due to the deployment of drones 104 to remove the methane within virtual fence 603, and the risk assessment of starting a fire by mining machines 101 located within and surrounding virtual fence 603, as shown in the following formula:

$$J = Max . {\sum }_{j = 1}^{N} \{ {u}_{j} ( {w}_{d r o n e} \cdot {E}_{d r o n e , j} - {w}_{d r o n e} \cdot {C}_{d r o n e , j} ) + {v}_{j} ( {w}_{vent} \cdot {E}_{v e n t , j} ) \}$$

where t_{drone,j}+t_{vent,j}≤t_{critical,j} and uⱼ+vⱼ≤1,
J is the cost function,
uⱼ is the binary decision variable for drone based methane removal,
vⱼ is the binary decision variable for ventilation based removal,
E_{drone,j} is the potential amount of methane diffusion at pixel ‘j,’ which is a function of methane intensity, location, required amount of oxygen and temperature and conversion efficiency of the catalyst,
E_{vent,j} is the potential amount of methane diffusion at pixel ‘j,’ which is a function of the type of ventilation,
C_{drone,j} is the cost of deploying drones with optimal oxygen, temperature and catalyst units for the hydrocarbon conversion process at pixel ‘j,’
w_{drone}, wᵥₑₙₜ is the weighing cost coefficient for hydrocarbon fuel and CO₂ conversion, respectively, as a function of greenhouse potential and carbon credits,
t_{drone,j}, t_{vent,j} is the time taken for methane diffusion by drone and ventilation-based system, respectively,
t_{critical,j} is the critical time available for methane diffusion at pixel ‘j’ considering the mining machine fire assessment,
w_{drone}·E_{drone,j} is the drone based methane conversion process,
w_{drone}·C_{drone,j} is the cost of deploying drones for the conversion process, and
wᵥₑₙₜ·E_{vent,j} is the ventilation based process.

In one embodiment, if removal strategy engine 505 determines to deploy drones 104 to remove the methane within virtual fence 603 via diffusion, then removal strategy engine 505 determines the optimal set of drones 104 to be deployed with an optimal number of methane catalyst units 307, temperature controller units 306, and oxygen supplier units 305 to minimize the time to diffuse the methane hotspots within virtual fence(s) 603 as shown in the following formula:

$$J = Min ( \frac{C i n {t}_{1}^{t}}{{C}_{1}} + \frac{C i n {t}_{2}^{t}}{{C}_{2}} + \frac{C i n {t}_{3}^{t}}{{C}_{3}} + … + \frac{C i n {t}_{n}^{t}}{{C}_{n}} )$$

where Cint is the methane intensity at hotspot (virtual fence 603) 1 at time t,
C₁ is the methane conversion/diffusion rate (either hydrocarbon fuel or CO₂) at hotspot (virtual fence 603) 1 at time t,

$${C}_{1} = f ( x 1 \cdot MCU , x 2 \cdot OSU , x 3 \cdot TCU ) ,$$

MCU is the methane catalyst unit 307,
OSU is the oxygen supplier unit 305,
TCU is the temperature controlling unit 306, and
x1, x2, and x3 are the number of selected units.

In one embodiment, the constraints for the above-described formula are the following:

$$\frac{{Cint}_{1}^{t}}{{C}_{1}} < t 1 , \frac{{Cint}_{2}^{t}}{{C}_{2}} < t 2 , … , \frac{{Cint}_{n}^{t}}{{C}_{n}} < t n$$

In one embodiment, removal strategy engine 505 optimizes the control parameters for drone-based methane removal. In one embodiment, since the control of temperature and oxygen supply needs to be very precise and subject to small changes, the temperature and oxygen are chosen in a matter that it is optimal for the next “K” steps as shown in the following formula:

$$J = {\sum }_{i = 1}^{N} {{w}_{{H}_{i}} ( {H}_{i}^{r} - {H}_{i}^{m} )}^{2} + {\sum }_{i = 1}^{N} {{w}_{{L}_{i}} ( {L}_{i}^{r} - {L}_{i}^{m} )}^{2} + {\sum }_{i = 1}^{N} {{w}_{u {H}_{i}} ( Δ u )}^{2} + {\sum }_{i = 1}^{N} {{w}_{v {L}_{i}} ( Δ v )}^{2} \\ subject to {H}^{min} \leq {H}_{i}^{m} \leq {H}^{max} and {L}^{min} \leq {L}_{i}^{m} \leq {L}^{max}$$

where J is the cost function over the receding horizon,
Hᵢ^{r} is the required temperature for the instant ‘i’ from the knowledge base,
Hᵢ^{m} is the measured temperature for the instant ‘i,’
Lᵢ^{r} is the required oxygen for the instant ‘i’ from the knowledge base,
Lᵢ^{m} is the measured oxygen for the instant ‘i,’
u, v are the temperature and oxygen controller variables, respectively,
w_{H}_{ᵢ}, w_{L}_{ᵢ} are the weighting coefficients for temperature and oxygen, respectively,
w_{uH}_{ᵢ}, w_{uL}_{ᵢ} are the penalizing coefficients for large changes in the temperature and supplied oxygen, respectively,
H^{max}, L^{max} are the maximum limits for temperature and oxygen, respectively, and
H^{min}, L^{min} are the minimum limits for temperature and oxygen, respectively.

In one embodiment, upon identifying the appropriate methane removal strategy, in step 907, removal strategy engine 505 of methane leak manger 102 implements the appropriate methane removal strategy for removing the methane within virtual fence 603.

Upon removing the methane from virtual fence 603 in such a manner that the concentration level of methane within virtual fence 603 has been reduced to a safe level (i.e., the level of concentration of methane in the air within virtual fence 603 is less than the threshold value), in step 908, scheduling engine 504 of methane leak manger 102 alters the work order schedule of mining machines 101, whose operations were reassigned to the alternative mining machines 101, such as to perform the operations as originally assigned to mining machines 101 prior to the detection of an unsafe level of methane to the extent that such operations were not completed by the alternative mining machines 101.

In this manner, methane leaks in mining operations are managed in a manner that prevents methane explosions with minimal impact on productivity.

Furthermore, the principles of the present disclosure improve the technology or technical field involving mining operations.

As discussed above, mining operations may involve the leakage of methane (CH₄), such as from coal seams and surrounding rock strata. For example, methane may be released from coal seams. When coal seams are fractured during mining, the methane trapped under pressure is released. In surface mines, drainage systems are a source of methane emissions. Furthermore, methane can seep out during the processing, storage, and transport of coal in underground mines. Additionally, methane can be released from abandoned mines. Methane is a greenhouse gas that is explosive when mixed with air so it poses a safety risk. An explosion may be set off by an electrostatic spark creating a huge increase in gas pressure. Such an electrostatic spark may be caused by the mining machines operating in the area where the concentration level of methane has reached an unsafe level. Such methane explosions can lead to mine evacuations, shutdowns, and stoppages, which can cause significant delays in production and loss of revenue. Unfortunately, there is not currently a means for managing methane leaks in mining operations in a manner that prevents methane explosions with minimal impact on productivity.

Embodiments of the present disclosure improve such technology by monitoring levels of concentration of methane in the air using methane sensors, such as at a mining facility. A mining facility, as used herein, is a place that extracts, mills, and processes minerals into a marketable form. It may include buildings, equipment, machinery, and other infrastructure. Upon identifying a level of concentration of methane in the air from a methane sensor that exceeds a threshold value, which may be user-designated, a virtual fence is created around the methane sensor to identify an area where the level of concentration of methane in the air exceeds the threshold value. A virtual fence, as used herein, refers to an invisible border, which marks the area where the level of concentration of methane in the air exceeds the threshold value. A methane removal strategy (e.g., deploying drones to remove methane within the virtual fence using diffusion or adsorption, using a ventilation-based system) is then developed for removing the methane within the virtual fence, such as based on the cost of deploying drones with oxygen, temperature, and catalyst units. Oxygen is used by drones to remove methane because it combines with methane to form carbon dioxide, a less potent greenhouse gas. Temperature is important for removing methane because it affects how the gas breaks down and the energy required for the process. For example, methane decomposition is an endothermic process (i.e., requires high temperature to break down the gas). Furthermore, the energy demand for a methane removal process is affected by the temperature at which the reaction occurs. Catalysts are needed to remove methane because they can accelerate the oxidation of methane by air, turning it into less harmful substances, such as carbon dioxide. Examples of catalysts include, but are not limited to, aluminosilicate minerals, palladium-based catalysts, mechanochemically prepared catalysts, etc. After developing a methane removal strategy for removing the methane within the virtual fence, the methane remove strategy is implemented. In this manner, methane leaks in mining operations are managed in a manner that prevents methane explosions with minimal impact on productivity. Furthermore, in this manner, there is an improvement in the technical field involving mining operations.

The technical solution provided by the present disclosure cannot be performed in the human mind or by a human using a pen and paper. That is, the technical solution provided by the present disclosure could not be accomplished in the human mind or by a human using a pen and paper in any reasonable amount of time and with any reasonable expectation of accuracy without the use of a computer.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A computer-implemented method for managing methane leaks, the method comprising:
monitoring levels of concentration of methane in air using methane sensors;
identifying a level of concentration of methane in said air from a first methane sensor that exceeds a first threshold value;
creating a virtual fence around said first methane sensor to identify an area where said level of concentration of methane in said air exceeds said first threshold value;
developing a methane removal strategy for removing methane within said virtual fence; and
implementing said methane removal strategy for removing methane within said virtual fence.

2. The method as recited in claim 1, wherein said methane removal strategy comprises one of the following in the group consisting of: deploying drones to remove methane within said virtual fence using diffusion or adsorption, and a ventilation-based system.

3. The method as recited in claim 1 further comprising;
identifying one or more mining machines within and surrounding said virtual fence with a risk of setting a fire that exceeds a second threshold value due to said level of concentration of methane in said air exceeding said first threshold value.

4. The method as recited in claim 3, wherein said one or more mining machines within and surrounding said virtual fence with said risk of setting said fire that exceeds said second threshold value are identified using digital twins of said one or more mining machines.

5. The method as recited in claim 3 further comprising:
altering a work order schedule of said identified one or more mining machines by having previously scheduled operations of said one or more identified mining machines being performed by one or more alternative mining machines.

6. The method as recited in claim 5, wherein said work order schedule is altered using a machine learning model, wherein said machine learning model considers functionality of said identified one or more mining machines and said one or more available alternative mining machines, risk of setting a fire by said one or more available alternative mining machines, and business criticality of said identified one or more mining machines and said one or more available alternative mining machines.

7. The method as recited in claim 5 further comprising:
altering said work order schedule of said identified one or more mining machines to perform one or more operations originally assigned to said identified one or more mining machines in response to said level of concentration of methane in said air being less than said first threshold value.

8. A computer program product for managing methane leaks, the computer program product comprising one or more computer readable storage mediums having program code embodied therewith, the program code comprising programming instructions for:
monitoring levels of concentration of methane in air using methane sensors;
identifying a level of concentration of methane in said air from a first methane sensor that exceeds a first threshold value;
creating a virtual fence around said first methane sensor to identify an area where said level of concentration of methane in said air exceeds said first threshold value;
developing a methane removal strategy for removing methane within said virtual fence; and
implementing said methane removal strategy for removing methane within said virtual fence.

9. The computer program product as recited in claim 8, wherein said methane removal strategy comprises one of the following in the group consisting of: deploying drones to remove methane within said virtual fence using diffusion or adsorption, and a ventilation-based system.

10. The computer program product as recited in claim 8, wherein the program code further comprises the programming instructions for:
identifying one or more mining machines within and surrounding said virtual fence with a risk of setting a fire that exceeds a second threshold value due to said level of concentration of methane in said air exceeding said first threshold value.

11. The computer program product as recited in claim 10, wherein said one or more mining machines within and surrounding said virtual fence with said risk of setting said fire that exceeds said second threshold value are identified using digital twins of said one or more mining machines.

12. The computer program product as recited in claim 10, wherein the program code further comprises the programming instructions for:
altering a work order schedule of said identified one or more mining machines by having previously scheduled operations of said one or more identified mining machines being performed by one or more alternative mining machines.

13. The computer program product as recited in claim 12, wherein said work order schedule is altered using a machine learning model, wherein said machine learning model considers functionality of said identified one or more mining machines and said one or more available alternative mining machines, risk of setting a fire by said one or more available alternative mining machines, and business criticality of said identified one or more mining machines and said one or more available alternative mining machines.

14. The computer program product as recited in claim 12, wherein the program code further comprises the programming instructions for:
altering said work order schedule of said identified one or more mining machines to perform one or more operations originally assigned to said identified one or more mining machines in response to said level of concentration of methane in said air being less than said first threshold value.

15. A system, comprising:
a memory for storing a computer program for managing methane leaks; and
a processor connected to the memory, wherein the processor is configured to execute program instructions of the computer program comprising:
monitoring levels of concentration of methane in air using methane sensors;
identifying a level of concentration of methane in said air from a first methane sensor that exceeds a first threshold value;
creating a virtual fence around said first methane sensor to identify an area where said level of concentration of methane in said air exceeds said first threshold value;
developing a methane removal strategy for removing methane within said virtual fence; and
implementing said methane removal strategy for removing methane within said virtual fence.

16. The system as recited in claim 15, wherein said methane removal strategy comprises one of the following in the group consisting of: deploying drones to remove methane within said virtual fence using diffusion or adsorption, and a ventilation-based system.

17. The system as recited in claim 15, wherein the program instructions of the computer program further comprise:
identifying one or more mining machines within and surrounding said virtual fence with a risk of setting a fire that exceeds a second threshold value due to said level of concentration of methane in said air exceeding said first threshold value.

18. The system as recited in claim 17, wherein said one or more mining machines within and surrounding said virtual fence with said risk of setting said fire that exceeds said second threshold value are identified using digital twins of said one or more mining machines.

19. The system as recited in claim 17, wherein the program instructions of the computer program further comprise:
altering a work order schedule of said identified one or more mining machines by having previously scheduled operations of said one or more identified mining machines being performed by one or more alternative mining machines.

20. The system as recited in claim 19, wherein said work order schedule is altered using a machine learning model, wherein said machine learning model considers functionality of said identified one or more mining machines and said one or more available alternative mining machines, risk of setting a fire by said one or more available alternative mining machines, and business criticality of said identified one or more mining machines and said one or more available alternative mining machines.
